(19) United States
(12) Patent Application Publication
(10) Pub. No.: US 2025/0264478 A1
(43) Pub. Date: Aug. 21, 2025

(54) IDENTIFICATION AND QUANTITATION OF HETERODIMERS IN MAB THERAPEUTICS

(71) Applicant: Regeneron Pharmaceuticals, Inc., Tarrytown, NY (US)

(72) Inventors: Wenjing Peng, Oakland, NJ (US); Yuetian Yan, Chappaqua, NY (US); Victoria Cotham, Lagrangeville, NY (US); Shunhai Wang, Scarsdale, NY (US); Ning Li, New Canaan, CT (US)

(21) Appl. No.: 19/042,366

(22) Filed: Jan. 31, 2025

Related U.S. Application Data
(60) Provisional application No. 63/548,580, filed on Feb. 1, 2024.
(60) Provisional application No. 63/689,443, filed on Aug. 30, 2024.
(60) Provisional application No. 63/693,571, filed on Sep. 11, 2024.

(51) Int. Cl. G01N 33/68; B01D 15/34; C12Q 1/34

(52) U.S. Cl. CPC G01N 33/6854; B01D 15/34; C12Q 1/34; G01N 33/6851; G01N 2333/98

(57) ABSTRACT

The present disclosure generally pertains to methods for characterizing dimers in an antibody co-formulation. In particular, the present disclosure pertains to the use of high-throughput size exclusion chromatography coupled to low-resolution mass spectrometry, deglycosylation, and immunodepletion to differentiate dimers of similar sizes.

### CROSS REFERENCE TO RELATED APPLICATIONS

This application claims priority to, and the benefit of, U.S. Provisional Application No. 63/548,580, which was filed on Feb. 1, 2024, U.S. Provisional Application No. 63/689,443, which was filed on Aug. 30, 2024, and U.S. Provisional Application No. 63/693,571, which was filed on Sep. 11, 2024, the disclosure of each of which is hereby incorporated by reference in its entirety.

### BACKGROUND

The strategy of co-formulating two or more therapeutic monoclonal antibodies (mAbs) and/or active proteins into one final drug product offers several advantages, including increased efficacy, overall reduced adverse events and improved patient convenience and compliance. However, the increased heterogeneity in a co-formulated drug product introduces additional challenges not only for formulation development, but also for analytical characterization. For example, the identification and quantitation of different dimer species present in a co-formulated drug product can be very challenging due to their low abundances and highly similar biophysical properties. Analytical methods for dimer analysis in mAb monotherapy products, such as size exclusion chromatography with UV detection, often lack the specificity that are required to differentiate various hetero- and homodimer species.

Protein biopharmaceutical products including co-formulated preparations should meet very high standards of purity. Thus, it may be important to monitor any impurities in the co-formulated drug at different stages of drug development, production, storage and handling. Analytical methods for assays used to characterize drug products should display sufficient accuracy and resolution to detect and quantify the desired product. Direct analysis can require isolation of the product in a sufficiently large amount for the assay, which is undesirable and has only been possible in selected cases.

Online coupling of SEC with direct MS detection under native conditions (nSEC-MS) has become increasingly popular for studying high molecular species of mAbs due to its high sensitivity, specificity, and ability to provide structural information. However, a more comprehensive evaluation of the validity, sensitivity, and general applicability of this nSEC-MS method is necessary before it can be widely adopted for heterodimer analysis to support mAb cocktail co-formulation development. In particular, factors that could potentially challenge the method's detection capability, such as the low abundances of heterodimers and dimers with close molecular weights that hinder their mass differentiation, should be carefully investigated and addressed.

Therefore, there remains a long felt need in the art for sensitive and efficient analytical methods and systems for characterizing dimer species present in co-formulated preparations.

### SUMMARY

The present disclosure provides methods for identifying and/or quantifying a multimer of interest from an antibody preparation, the methods comprising:
deglycosylating the antibody preparation to form a deglycosylated sample;
separating the deglycosylated sample by size exclusion chromatography separation to form a separated sample; and
analyzing the separated sample by nano-electrospray ionization (NSI) mass spectrometry analysis to identify and/or quantify the multimer of interest.

This disclosure provides methods for identifying and/or quantifying at least one polypeptide or polypeptide multimer of interest. In some exemplary embodiments, the methods can comprise: (a) subjecting a sample including at least one polypeptide or polypeptide multimer of interest to deglycosylation to form a deglycosylated sample; (b) subjecting the deglycosylated sample to size exclusion chromatography separation to form a separated sample; and (c) subjecting the separated sample to mass spectrometry analysis to identify and/or quantify the at least one polypeptide or polypeptide multimer of interest, wherein the method is performed under native conditions.

In one aspect, the size exclusion chromatography system and the mass spectrometer are connected by a splitter. In a specific aspect, the three-way splitter splits an eluate from the size exclusion chromatography system between the mass spectrometer and an ultraviolet detection system.

In one aspect, the at least one polypeptide or polypeptide multimer of interest comprises a homodimer and/or a heterodimer. In another aspect, the at least one polypeptide or polypeptide multimer of interest is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof.

In one aspect, the sample is a co-formulation sample. In another aspect, the sample includes two, three, or more antibodies. In another aspect, the concentration of the antibody is about 0.5 mg/mL to about 100 mg/mL. In yet another aspect, the concentration of the antibody is about 0.5 mg/mL to about 5 mg/mL.

In one aspect, the mass spectrometry analysis comprises nano-electrospray ionization mass spectrometry.

In one aspect, at least two polypeptides or polypeptide multimers of interest are identified or quantified, and the identifying and/or quantifying includes determining a relative abundance of each of the at least two polypeptides or polypeptide multimers of interest. In a specific aspect, the method further comprises quantifying an absolute percent of the at least two polypeptides or polypeptide multimers of interest in the sample. In a more specific aspect, the quantifying comprises multiplying the relative abundance by a total percent of the at least two polypeptides or polypeptide multimers of interest in the sample as measured using ultraviolet detection.

In one aspect, the size exclusion chromatography separation is performed with a flow rate of 0.2 mL/min. In one aspect, a mobile phase for the size exclusion chromatography separation comprises ammonium acetate, ammonium bicarbonate, or ammonium formate, or combinations thereof. In another aspect, a mobile phase for the size exclusion chromatography separation comprises ammonium acetate. In another aspect, the concentration of ammonium acetate is 150 mM.

In one aspect, the size exclusion chromatography is high-throughput size exclusion chromatography. In another aspect, the high-throughput size exclusion chromatography separation is performed with a 150 mm size exclusion chromatography column.

In another aspect, the mass spectrometry is low-resolution mass spectrometry. the resolution setting for the low-resolution mass spectrometry is 6,250 (at m/z=200).

In one aspect, the sample is an immuno-depleted sample. In a specific aspect, the immuno-depleted sample is formed by subjecting the sample to at least one immunoprecipitation step comprising at least one immunoprecipitation antibody that binds to at least one molecule in the sample that is not the at least one polypeptide or polypeptide multimer of interest.

In one aspect, the sample is a stressed sample. In a specific aspect, the stress comprises storing the sample at a temperature from −80° C. to 40° C., about 5° C., about 25° C., or about 40° C. In a more specific aspect, the sample is stored at the temperature from 0 to 6 months, about 1 month, about 3 months, or about 6 months.

In one aspect, subjecting a sample including at least one polypeptide or polypeptide multimer of interest to deglycosylation comprises contacting the sample to PNGase F.

This disclosure additionally provides methods for identifying and/or quantitating at least one antibody dimer in an antibody co-formulation. In some exemplary embodiments, the methods can comprise (a) subjecting an antibody co-formulation sample to deglycosylation to form a deglycosylated sample; (b) subjecting the deglycosylated sample to high-throughput size exclusion chromatography separation to form a separated sample; and (c) subjecting the separated sample to low-resolution mass spectrometry analysis to identify and/or quantify the at least one antibody dimer, wherein the method is performed under native conditions and at least one splitter with at least three paths is used to couple the size exclusion chromatography system with the mass spectrometer and with an ultraviolet detector.

In one aspect, the antibody co-formulation sample comprises at least two antibodies or at least three antibodies. In another aspect, the concentration of the antibody is about 0.5 mg/mL to about 100 mg/mL. In yet another aspect, the concentration of the antibody is about 0.5 mg/mL to about 5 mg/mL.

In one aspect, the antibody co-formulation sample comprises at least one homodimer and at least one heterodimer. In another aspect, the antibody co-formulation sample comprises at least two dimers, at least three dimers, at least four dimers, at least five dimers, or at least six dimers.

In one aspect, subjecting a sample including at least one polypeptide or polypeptide multimer of interest to deglycosylation comprises contacting the sample to PNGase F.

In one aspect, the antibody co-formulation sample is an immuno-depleted sample. In a specific aspect, the immuno-depleted sample is formed by subjecting the sample to at least one immunoprecipitation step comprising at least one immunoprecipitation antibody that binds to at least one antibody in the sample.

In one aspect, the high-throughput size exclusion chromatography separation is performed with a flow rate of 0.6 mL/min. In another aspect, the high-throughput size exclusion chromatography separation is performed with a 150 mm size exclusion chromatography column.

In one aspect, a mobile phase for the high-throughput size exclusion chromatography separation comprises ammonium acetate, ammonium bicarbonate, or ammonium formate, or combinations thereof. In another aspect, a mobile phase for the high-throughput size exclusion chromatography separation comprises ammonium acetate. In another aspect, the concentration of ammonium acetate is 150 mM.

In one aspect, the low-resolution mass spectrometry analysis comprises nano-electrospray ionization mass spectrometry. In another aspect, the resolution setting for the low-resolution mass spectrometry analysis is 6,250 (at m/z=200).

These, and other, aspects of the present disclosure will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 shows a comparison of size-exclusion chromatography-ultraviolet (SEC-UV) and native size-exclusion chromatography-ultraviolet/mass spectrometry (native SEC-MS).

FIG. 2 shows a schematic of native SEC-UV/MS workflow.

FIG. 3 shows a comparison of relative abundance of high molecular weight species from different FDC samples (N=80) using mobile phase vs MS-compatible mobile phase, according to an exemplary embodiment.

FIG. 4A shows raw mass spectra (top) and the corresponding deconvoluted mass spectra (bottom) of the dimer species from a two-mAb cocktail sample (60 mg/mL Ab1 and 60 mg/mL Ab2 stored at 5° C. for 3 months) without PNGase F treatment, according to an exemplary embodiment. The zoomed-in view of the charge state z=+35 in each raw mass spectrum is shown as an inset.

FIG. 4B shows raw mass spectra (top) and the corresponding deconvoluted mass spectra (bottom) of the dimer species from a two-mAb cocktail sample (60 mg/mL Ab1 and 60 mg/mL Ab2 stored at 5° C. for 3 months) with PNGase F treatment, according to an exemplary embodiment. The zoomed-in view of the charge state z=+35 in each raw mass spectrum is shown as an inset.

FIG. 5A shows deconvoluted MS spectra of the dimer species from a two-mAb cocktail sample after native deglycosylation, according to an exemplary embodiment. The upper trace represents the dimers detected in the Ab1/Ab2 co-formulated sample (60 mg/mL Ab1 and 60 mg/mL Ab2 and stored at 5° C. for 3 months), while the lower trace represents the dimers detected in the freshly mixed Ab1/Ab2 standards.

FIG. 5B shows deconvoluted MS spectra of the dimer species from a three-mAb cocktail sample after native deglycosylation, according to an exemplary embodiment. The upper trace represents the dimers detected in the Ab1/Ab2/Ab3 co-formulated sample (33.3 mg/mL Ab1, 33.3 mg/mL Ab2, and 33.3 mg/mL Ab3), while the lower trace represents the dimers detected in the freshly mixed Ab1/Ab2/Ab3 standards.

FIG. 6 shows UV trace of SEC chromatogram of a two-mAb cocktail sample stored at 40° C. for different time points (0, 1, and 3 min), according to an exemplary embodiment.

FIG. 7 shows the growth of heterodimer abundance (%) in a two-mAb cocktail sample (60 mg/mL Ab1 and 60 mg/mL Ab2) under different storage/stress temperatures, according to an exemplary embodiment. The insets show the distributions of hetero- and homodimers at TO and different storage/thermal stress end points.

FIG. 8 shows a comparison of high-throughput-nSEC (HT-nSEC) and nSEC, according to an exemplary embodiment.

FIG. 9 shows a comparison of HT-nSEC-MS method and nSEC-MS method in analyzing a stressed FDC sample (60 mg/mL Ab1 and 60 mg/mL Ab2 stored at 25° C. for 20 months), according to an exemplary embodiment. The inset shows the deconvoluted mass spectrum of the dimer peak and MS-based quantitation of the dimer distribution from each method.

FIG. 10A shows a comparison of raw mass spectra (left) and deconvoluted mass spectra (right) obtained at R=12,500 and R=6,250 for dimer species in a two-mAb cocktail sample (40 mg/mL Ab6 and 50 mg/mL Ab7, according to an exemplary embodiment. The zoomed-in view of the charge state z=+36 in each raw mass spectrum is shown as an inset. MS-based quantitation of the dimer distribution is indicated in each deconvoluted mass spectrum.

FIG. 10B shows a comparison of raw mass spectra (left) and deconvoluted mass spectra (right) obtained at R=12,500 and R=6,250 for dimer species in an IV compatibility sample (1 mg/mL Ab6 and 5 mg/mL Ab7), according to an exemplary embodiment. The zoomed-in view of the charge state z=+36 in each raw mass spectrum is shown as an inset. MS-based quantitation of the dimer distribution is indicated in each deconvoluted mass spectrum.

FIG. 11A shows a schematic of the immunodepletion-assisted SEC-MS (ID-SEC-MS) workflow for dimer quantitation for a stressed three-mAb cocktail sample (60 mg/mL Ab8, 60 mg/mL Ab9, and 60 mg/mL Ab10 stored at 25° C. for 1 month), according to an exemplary embodiment.

FIG. 11B illustrates a strategy for quantitating species of dimers in a sample, according to an exemplary embodiment.

FIG. 11C illustrates calculations for quantitating species of dimers in a sample, according to an exemplary embodiment.

FIG. 12 shows deconvoluted mass spectra of the dimers in the untreated cocktail sample (top) and immunodepleted cocktail sample using anti-Ab9 antibody (bottom), and the relative abundances of all six dimer species, according to an exemplary embodiment.

FIG. 13 shows the UV trace of SEC chromatogram of a three-mAb cocktail sample (60 mg/mL Ab8, 60 mg/mL Ab9, and 60 mg/mL Ab10 stored at 25° C. for 1 month) before and after depletion, according to an exemplary embodiment.

### DETAILED DESCRIPTION

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. As used herein, the term polypeptide includes proteins, variants thereof, fragments thereof, and peptides, whether synthetic, naturally occurring, or derived from a larger polypeptide, for example through digestion or truncation. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like.

Proteins of interest or polypeptides of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation. Biotechnology and Genetic Engineering Reviews, 2012, 28, 147-176, the entire teachings of which are herein incorporated by reference).

In some exemplary aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

As used herein, the term “therapeutic protein” refers to any protein that can be administered to a subject for the treatment of a disease or disorder. A therapeutic protein may be any protein with a pharmacological effect, for example, an antibody, a soluble receptor, an antibody-drug conjugate, an antigen-binding protein, or an enzyme. In some exemplary embodiments, the therapeutic protein can be a monoclonal antibody. In some exemplary embodiments, two or more therapeutic proteins may be present in the same sample. Multiple therapeutic proteins may be co-administered in order to achieve a pharmacological effect, for example, to prevent viral escape due to mutation of a target virus.

As used herein, the term “antibody cocktail” or “co-formulation” refers to co-administered therapeutic proteins comprising at least two therapeutic antibodies. In some exemplary embodiments, the co-formulation can comprise at least three therapeutic antibodies. This co-formulation can be used to treat, prevent, or ameliorate a certain disease condition by targeting different molecular targets and obtain an overall improved medical condition of the patient due to additive and/or synergistic effects as compared to the single dug(s) alone (Mueller, C., Altenburger, U., and Mohl, S., Challenges for the pharmaceutical technical development of protein coformulations. Journal of Pharmacy and Pharmacology, 2017, 70, 666-674). Some of the advantages include increased efficacy compared to a single drug, overall reduction of adverse event, improvement of patient convenience and compliance (increased patient adherence, simplified patient guidance and education), reduction in health care costs (manufacture and purchase), easier supply processes, and new product opportunities within life-cycle management of existing marketed products.

Therapeutic monoclonal antibodies (mAb) have demonstrated remarkable success in the treatment of various diseases, attributable to their exceptional specificity, efficacy, stability, and safety profiles (Krieg, D. et al., It is Never Too Late for a Cocktail-Development and Analytical Characterization of Fixed-dose Antibody Combinations. J Pharm Sci., 2022 111(8), 2149-2157; Chiavenna, S. M. et al., State of the art in anti-cancer mAbs. J Biomed Sci., 2017, 24(1), 15; Kaplon, H. et al., Antibodies to watch in 2023. MAbs, 2023, 15(1), 2153410). In recent developments, combinations of two or more mAbs as a cocktail treatment have attracted significant interest due to their improved therapeutic outcomes, such as enhanced efficacy from interacting with different targets (or different epitopes within a target) and reduced resistance from the pathogen or cancer cells (Chauhan, V. M. et al., Advancements in the co-formulation of biologic therapeutics. J Control Release, 2020, 327, 397-405; Copin, R. et al., The monoclonal antibody combination REGEN-COV protects against SARS-COV-2 mutational escape in preclinical and human studies. Cell, 2021, 184 (15), 3949-3961; Baselga, J. et al., Pertuzumab plus trastuzumab plus docetaxel for metastatic breast cancer. N Engl J Med., 2012, 366 (2), 109-19). Additionally, the mAb cocktail co-formulation approach, when compared to sequential or co-administration approaches, offer advantages in reduced risk of medical error (Krieg, D. et al.) and better patient compliance and convenience (Chauhan, V. M. et al.).

However, despite the benefits conferred by mAb cocktails, they also introduce additional challenges in clinical and regulatory procedures, process developments, and analytical quality control (Tan, A. R. et al. Fixed-dose combination of pertuzumab and trastuzumab for subcutaneous injection plus chemotherapy in HER2-positive early breast cancer (FeDeriCa): a randomised, open-label, multicentre, non-inferiority, phase 3 study. Lancet Oncol., 2021, 22 (1), 85-97, USFDA, Codevelopment of Two or More New Investigational Drugs for Use in Combination. 2013; Mueller, C. et al., Challenges for the pharmaceutical technical development of protein coformulations. J Pharm Pharmacol., 2018, 70 (5), 666-674). One of the primary challenges in the development of antibody cocktail co-formulation arises from the need to identify a common formulation condition that can adequately maintain the stability and control the variant profiles of all constituent mAbs in the drug sample. Among the potential variant forms, heterodimers, arising from intermolecular interactions between co-formulated mAbs, are particularly important to monitor, as they are unique to the co-formulated drug product (Mueller, C. et al.). In fact, protein aggregates have been reported to potentially mediate adverse events, including increased immunogenicity, reduced product efficacy, and enhanced hypersensitivity (Nabhan, M. et al., Immunogenicity of Bioproducts: Cellular Models to Evaluate the Impact of Therapeutic Antibody Aggregates. Front Immunol., 2020, 11, 725; Moussa, E. M. et al., Immunogenicity of Therapeutic Protein Aggregates. J Pharm Sci., 2016, 105 (2), 417-430; Rosenberg, A. S., Effects of protein aggregates: an immunologic perspective. Aaps j., 2016, 8 (3), E501-7). Therefore, the heterodimer, as a particular format of protein aggregates in the antibody cocktails, is regarded as a critical quality attribute and must be carefully characterized and monitored throughout the development of therapeutic mAb cocktail products.

In recent years, numerous technologies and methodologies have been developed for size variant analysis to support the development of protein therapeutics. A few commonly applied techniques include sedimentation velocity analytical ultracentrifugation (SV-AUC) (Zhou, M. et al., Application of Affinity-Capture Self-Interaction Nanoparticle Spectroscopy in Predicting Protein Stability, Especially for Co-Formulated Antibodies. Pharm Res., 2021, 38 (4), 721-732; Yang, D. et al., Weak IgG self- and hetero-association characterized by fluorescence analytical ultracentrifugation. Protein Sci., 2018, 27 (7), 1334-1348), asymmetrical flow field-flow fractionation (AF4) (Hawe, A. et al., Asymmetrical flow field-flow fractionation method for the analysis of submicron protein aggregates. J Pharm Sci. 2012, 101 (11), 4129-39; Litzén, A. et al., Separation and quantitation of monoclonal antibody aggregates by asymmetrical flow field-flow fractionation and comparison to gel permeation chromatography. Anal Biochem., 1993, 212 (2), 469-80), and size exclusion chromatography (SEC) (Yan, Y. et al., Post-Column Denaturation-Assisted Native Size-Exclusion Chromatography-Mass Spectrometry for Rapid and In-Depth Characterization of High Molecular Weight Variants in Therapeutic Monoclonal Antibodies. J Am Soc Mass Spectrom. 2021, 32 (12), 2885-2894; D'Atri, V. et al., Size exclusion chromatography of biopharmaceutical products: From current practices for proteins to emerging trends for viral vectors, nucleic acids and lipid nanoparticles. J Chromatogr A., 2024, 1722, 464862) due to their excellent resolving power of size variants and quantitative performance. In particular, SEC with ultraviolet (UV) detection is routinely employed as a batch release assay for monitoring the levels and profiles of high molecular weight (HMW) species in therapeutic mAb products (See, Hong, P. et al., Size-Exclusion Chromatography for the Analysis of Protein Biotherapeutics and their Aggregates. J Liq Chromatogr Relat Technol., 2012, 35 (20), 2923-2950; Zölls, S. et al., Particles in therapeutic protein formulations, Part 1: overview of analytical methods. J Pharm Sci., 2012, 101 (3), 914-35). These separation techniques, however, face significant challenges when analyzing the dimer species present in mAb cocktails. This is due to the highly heterogenous dimer compositions (e.g., homodimers and heterodimers) in the cocktail samples and their similar biophysical properties, which hinder efficient analytical separation. In addition, commonly used detection techniques such as UV, fluorescence (FLR), multi-angle light scattering (MALS) are not specific enough to differentiate various hetero- and homodimer species and to allow for their individual quantitation (Yan, Y. et al., 2013; D'Atri, V. et al.; Lu, C. et al., Characterization of monoclonal antibody size variants containing extra light chains. MAbs, 2013, 5 (1), 102-13; den Engelsman, J. et al., Strategies for the assessment of protein aggregates in pharmaceutical biotech product development. Pharm Res., 2011, 28 (4), 920-33).

Over the past few years, online coupling of SEC with direct MS detection under native conditions (nSEC-MS) has become increasingly popular for studying mAb HMW species due to its high sensitivity, specificity, and ability to provide structural information (See, Ladwig, P. M. et al., Mass Spectrometry Approaches for Identification and Quantitation of Therapeutic Monoclonal Antibodies in the Clinical Laboratory. Clin Vaccine Immunol., 2017, 24 (5), e00545-16; Dai, J. and Ji, C. In-depth size and charge variants characterization of monoclonal antibody with native mass spectrometry. Anal Chim Acta., 2023, 1265, 341360, and Murisier, A. et al., Direct coupling of size exclusion chromatography and mass spectrometry for the characterization of complex monoclonal antibody products. J Sep Sci., 2022, 45 (12), 1997-2007). Using MS-compatible mobile phases (e.g., ammonium acetate) that preserve the native conformations and inter-molecular interactions of mAb dimers, nSEC-MS is particularly useful for enabling simultaneous identification and quantitation of multiple dimer species from a mAb cocktail sample based on accurate mass measurements. In a previous report, the feasibility of applying nSEC-MS to differentiate and quantitate a heterodimer from two homodimers in a two-mAb cocktail sample was demonstrated (Yan, Y. et al., 2013). However, a more comprehensive evaluation of the validity, sensitivity, and general applicability of this nSEC-MS method is necessary before it can be widely adopted for heterodimer analysis to support mAb cocktail co-formulation development. In particular, factors that could potentially challenge the method's detection capability, such as the low abundances of heterodimers (common to unstressed samples) and dimers with close molecular weights that hinder their mass differentiation (common to three-mAb cocktails), should be carefully investigated and addressed. The antibody preparation may be stressed prior to deglycosylaton by storing the antibody preparation at a temperature from −80° C. to 40° C. The antibody preparation may be stored at the temperature from 0 to about 6 months. In an exemplary embodiment, the stress comprises storing the antibody preparation at a temperature from −80° C. to 40° C., about 5° C., about 25° C., or about 40° C. In an exemplary embodiment, the antibody preparation is stored at the temperature from 0 to 6 months, about 1 month, about 3 months, or about 6 months.

Alternative to nSEC-MS, a multiple dimensional liquid chromatography coupled to MS detection (mD-LC-MS/MS) method was recently introduced, reporting its application in detecting covalently associated heterodimers in co-formulated mAb cocktails (Kuhne, F. et al. Identification of Hetero-aggregates in Antibody Co-formulations by Multi-dimensional Liquid Chromatography Coupled to Mass Spectrometry. Anal Chem., 2023, 95 (4), 2203-2212). Specifically, the covalent dimers from the cocktail sample was fractionated using reversed-phase (RP) LC (1st dimension), followed by online trypsin digestion (2nd dimension), and bottom-up analysis by RPLC-MS/MS (3rd dimension). Subsequently, crosslinked dipeptides consisting of unique peptides from each mAb were used as surrogates to compare the relative abundances of each hetero- and homodimer. However, a significant drawback to this method is that it cannot provide a complete assessment of the dimer species from the mAb cocktail samples, as the non-covalent dimers cannot be preserved and analyzed under denaturing conditions. Therefore, there remains a significant unmet need for advanced analytical methods to characterize the heterogeneous dimer species present in co-formulated mAb cocktails, which are crucial for their successful development.

A nSEC-MS-based method and associated analytical strategies were developed and evaluated for heterodimer identification and quantitation in co-formulated mAb cocktails. First, the suitability of using an MS-compatible SEC mobile phase was assessed by comparing its quantitative performance with SEC-UV methods that use non-volatile buffers, in testing a large set of mAb samples. Further, multiple strategies, including native deglycosylation treatment, low-resolution (LR) MS data acquisition, and a high-throughput SEC-MS method, were developed and demonstrated to significantly improve the method sensitivity and throughput. Lastly, to cope with the increasing complexity of the three-mAb cocktail samples where mass detection alone was insufficient to resolve all dimers, an immunodepletion-assisted SEC-MS method was developed to interpret the convoluted dimer spectra and achieve individual quantitation of all six hetero- and homodimers. Together, it was demonstrated that the developed nSEC-MS method and the associated analytical strategies provide a highly effective tool for the identification and quantitation of heterodimers in co-formulated mAb cocktails and therefore, holds great promise in supporting the development of next generation mAb cocktail therapies.

The strategy of co-formulating two or more therapeutic mAbs into one final drug product offers several advantages, including increased efficacy, overall reduced adverse events and improved patient convenience and compliance. In addition to challenges with formulation development, such as choosing excipients and conditions to compromise between the needs of multiple therapeutic proteins, co-formulated drugs also present a significant challenge for analytical characterization. For example, differentiation and quantitation of different dimer forms present in a co-formulated drug during storage or stressed conditions can be extremely challenging.

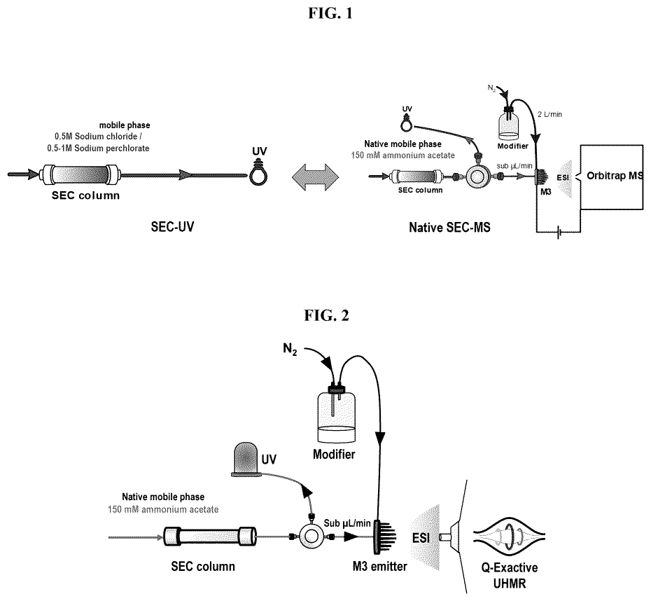

Challenges in heterodimer characterization in co-formulated studies include their low abundances and similar biophysical properties that result in inefficient separation by native liquid chromatography methods. Size exclusion chromatography with ultraviolet detection (SEC-UV) often cannot distinguish homodimers from heterodimers. SEC coupled with mass spectrometry (SEC-MS) often cannot resolve homodimers and heterodimers when the masses of co-formulated mAbs are too similar. A high throughput is also needed for analysis of large sets of samples. In order to address the long-felt needs in resolving low-abundance, biophysically similar molecules such as dimers of a mAb co-formulation, optimized SEC-MS-based methods were developed in combination with novel sample handling approaches. An MS-compatible buffer was used for direct hyphenation of SEC to MS in native SEC-MS (nSEC-MS) dimer quantitation (FIG. 1). MS complexity was reduced using a deglycosylation step. The method was validated by quantitating heterodimer formation under different thermal stress conditions. Furthermore, a high-throughput method was developed to facilitate large-scale heterodimer analysis. A low-resolution MS method was used to improve heterodimer quantitation. Additionally, an immunoprecipitation-assisted approach was developed to further improve heterodimer quantitation even for co-formulations with mAbs of very similar masses.

The present disclosure provides methods for identifying and/or quantifying a multimer of interest from an antibody preparation, the methods comprising:
deglycosylating the antibody preparation to form a deglycosylated sample;
separating the deglycosylated sample by size exclusion chromatography separation to form a separated sample; and
analyzing the separated sample by nano-electrospray ionization (NSI) mass spectrometry analysis to identify and/or quantify the multimer of interest.

The method may further comprise quantifying a total percent abundance of the multimer of interest in the antibody preparation. To calculate the total dimer abundance in each sample, the UV-based peak area of the dimer can be compared to the combined peak areas of both dimer and monomer peaks. The relative abundance (distribution) of each dimer species within the total dimer peak of a mAb cocktail sample can be calculated using its integrated mass peak area and compared against the summed mass peak areas of all dimers. The absolute abundance of each dimer in the cocktail sample can be calculated by multiplying the UV-based total dimer abundance with the MS-determined relative abundance of each dimer.

Heterodimers form via inter-molecular interactions between two different mAbs upon co-formulation or co-administration, and are important quality attributes to monitor during the development of therapeutic mAb cocktails. The present disclosure provides a widely applicable nSEC-MS method for effective dimer analysis in mAb cocktails. Tailored sample treatment strategies were developed to facilitate dimer differentiation and quantitation in both two-mAb and three-mAb cocktails.

Without wishing to be bound by theory, due to the mass heterogeneity originated from the N-linked glycans, dimers exhibit a broad cluster of mass peaks, leading to notable mass overlaps with each other. If a heterodimer exhibits a molecular weight that falls right between the two homodimers, its mass profile cannot be clearly resolved from either homodimer, rendering MS-based quantitation challenging. To address this challenge, PNGase F-mediated deglycosylation treatment removes the Fc N-glycans and converts various mAb glycoforms into a single deglycosylated population. To preserve the non-covalent dimers that may be present in the sample, the deglycosylation reaction is conducted under mild conditions without any denaturants or surfactants. After the treatment, all dimer species exhibit distinct mass profiles that are sufficiently resolved from each other, allowing for unambiguous mass peak integration for individual dimer quantitation. In addition to the enhanced mass separation, a concomitant improvement in MS intensity, signal-to-noise ratio (S/N), and spectral quality are also observed for the deglycosylated sample, due to the signal consolidation from various mAb glycoforms, as a result of N-glycan removal. Thus, a deglycosylation step improves the differentiation and quantitation of hetero- and homodimers by reducing the mass heterogeneity from the Fc N-glycans. Deglycosylation does not cause artifactual dimer formation, thereby maintaining the quantitation accuracy.

Without wishing to be bound by theory, a positive correlation between the heterodimer growth rate and the storage/stress temperature can be observed, with the fastest growth at 40° C. (an average of 0.27% per month), a moderate growth at 25° C. (an average of 0.08% per month), and the slowest growth at the 5° C. storage conditions (an average of 0.03% per month). Comparing the relative distribution of hetero- and homodimers in the deconvoluted mass spectra, the relative abundance of heterodimers is significantly higher in the stressed samples (e.g., 25° C. at 6-month and 40° C. at 3-month) than in the control sample (TO). This suggests a higher propensity of heterodimer formation than homodimer under thermal stress conditions and highlights the benefit of monitoring heterodimer formation during therapeutic mAb cocktail co-formulation development.

Heterodimers form after the mixing of individual mAb samples, and therefore their relative abundance is low in fixed-dose co-formulation (FDC) samples under normal storage or mild stress conditions. In addition, the absolute abundance of the heterodimers is dependent on the concentrations of the individual mAbs. For example, intravenous (IV) compatibility studies may be performed during formulation development to support mAb cocktail co-administration. In such studies, individual mAbs are diluted and mixed at low concentration ranges which lead to low levels of heterodimers and pose challenges for MS detection. Through dimer proteoform integration, the low-resolution (LR)-SEC-MS method (R=6,250) provides a significant improvement in both overall signal intensity and S/N ratio for dimer species, and allows for confident spectral deconvolution.

While a two-mAb cocktail sample may contain up to three dimer species (i.e., two homodimers and one heterodimer), a three-mAb cocktail sample could contain a total of six different dimer species (i.e., three homodimers and three heterodimers). An immunodepletion-assisted SEC-MS (ID-SEC-MS) workflow uses resins coated with an anti-drug Ab that specifically binds to one of the mAb components in the cocktail, thereby removing multiple dimer species containing this mAb. This treatment leads to simplified dimer complexity and facilitates MS-based dimer differentiation and quantitation. When combined with the results from the untreated sample, the abundance of the removed dimer species can also be back-calculated. When applied to a three-mAb cocktail sample, the individual dimer abundances of all six dimer species in the three-mAb cocktail sample can be determined by this immunodepletion-assisted nSEC-MS (ID-SEC-MS) method. Further, this ID-SEC-MS method may also be applicable to mAb cocktails comprising of more than three mAbs.

These methods represent novel analytical approaches to enable unambiguous and sensitive evaluation of heterodimers directly from the mAb cocktail drug product samples and, therefore, are of utility to support mAb cocktail therapy development.

In some aspects, a sample including two or more polypeptides, such as an antibody co-formulation sample, may comprise multimeric species composed of at least one of the two or more polypeptides, including dimers. In one aspect, the dimers may be homodimers of one polypeptide species or heterodimers of two different polypeptide species. The multimer of interest may comprise a homodimer and/or a heterodimer.

The monomer of the multimer of interest may comprise an antibody, an antibody fragment, a fusion protein, a receptor fusion protein, an antibody-derived protein, an antigen-binding protein, or a variant or fragment thereof.

The antibody preparation may comprise two or more antibodies.

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a Fc/2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen.

An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Fan, G., Wang, Z., and Hao, M., Bispecific antibodies and their applications. Journal Of Hematology & Oncology, 2015, 8, 130; Müller, D., and Kontermann, R. E., Bispecific Antibodies. Handbook Of Therapeutic Antibodies, 2014, 265-310, the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the systems and methods disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some aspects, the concentration of an antibody in a co-formulation is about 0.5 mg/mL to about 100 mg/mL, about 0.5 mg/mL to about 5 mg/mL, about 50 mg/mL to about 100 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. The concentration of the two or more antibodies may be about 0.5 mg/mL to about 100 mg/mL.

As used herein, a “sample” can be obtained from any step of a bioprocess, such as cell culture fluid (CCF), harvested cell culture fluid (HCCF), any step in the downstream processing, final concentrated pool (FCP), drug substance (DS), or a drug product (DP) comprising the final formulated product. In some specific aspects, the sample can be selected from any step of the downstream process of clarification, chromatographic production, or filtration. In some specific exemplary aspects, the drug product can be selected from manufactured drug product in the clinic, shipping, storage, or handling.

In some exemplary aspects, the sample is a biological sample. As used herein, the term “biological sample” refers to a sample taken from a living organism, for example a human or non-human mammal. A biological sample may comprise or consist of, for example, whole blood, plasma, serum, saliva, tears, semen, cheek tissue, organ tissue, urine, feces, skin, or hair. A sample may be taken from a patient, for example, a clinical sample. In some exemplary aspects, a sample may be taken from a non-human animal, for example, a preclinical sample. In some exemplary aspects, a sample may be taken from a non-human animal subjected to gene therapy in order to produce at least one protein of interest or polypeptide of interest that may be included in the sample. In some aspects, a sample is a further processed form of any of the aforementioned examples of samples.

The following identifies and describes proteins made in cell culture that can be produced and/or characterized according to the present disclosures. Cells comprising the requisite DNA encoding these proteins can be cultured for production according to the present disclosures.

In some exemplary aspects, the protein of interest or polypeptide of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

For example, for antibody production, some embodiments are amenable for research and production use for diagnostics and therapeutics based on all major antibody classes, namely IgG, IgA, IgM, IgD, and IgE. IgG is a preferred class, and includes subclasses IgG1 (including IgG1λ and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-DII4 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or U.S. Pat. No. 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-ILIR antibody, an interleukin 4 receptor antibody (e.g., an anti-IL4R antibody as described in U.S. Pat. App. Pub. No. US2014/0271681A1 or U.S. Pat. No. 8,735,095 or U.S. Pat. No. 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-COV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-COV antibody), an anti-COVID-19 antibody (e.g., an anti-SARS-COV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3× anti-CD20 bispecific antibody (as described in U.S. Pat. App. Pub. Nos. US2014/0088295A1 and US20150266966A1), an anti-CD3× anti-Mucin 16 bispecific antibody (e.g., an anti-CD3× anti-Muc16 bispecific antibody), an anti-CD3×BCMA bispecific antibody, and an anti-CD3× anti-Prostate-specific membrane antigen bispecific antibody (e.g., an anti-CD3× anti-PSMA bispecific antibody). See also U.S. Patent Publication No. US 2019/0285580 A1. Also included are a Met×Met antibody, an agonist antibody to NPR1, an LEPR agonist antibody, a MUC16×CD28 antibody, a GITR antibody, an IL-2Rg antibody, an EGFRxCD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies can be cultured according to the disclosures. In some aspects, the protein of interest or polypeptide of interest is selected from the group consisting of alirocumab, atoltivimab, maftivimab, odesivimab, odesivimab-ebgn, casirivimab, imdevimab, cemplimab and cemplimab-rwlc (human igg4 monoclonal antibody that binds to pd-1), sarilumab, fasinumab, nesvacumab, dupilumab (human monoclonal antibody of the igg4 subclass that binds to the il-4r alpha (α) subunit and thereby inhibits interleukin 4 (il-4) and interleukin 13 (il-13) signaling), trevogrumab, evinacumab, evinacumab-dgnb, fianlimab, garetosmab, itepekimab, odrononextamab, pozelimab, rinucumab, and modifications, truncations, and variations thereof.

Additional exemplary antibodies include ravulizumab-cwvz, abciximab, adalimumab, adalimumab-atto, ado-trastuzumab, alemtuzumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cetuximab, denosumab, dinutuximab, durvalumab, eculizumab, elotuzumab, emicizumab-kxwh, emtansine alirocumab, evolocumab, golimumab, guselkumab, ibritumomab tiuxetan, idarucizumab, infliximab, infliximab-abda, infliximab-dyyb, ipilimumab, ixekizumab, mepolizumab, necitumumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, secukinumab, siltuximab, tocilizumab, trastuzumab, ustekinumab, and vedolizumab.

In addition to next generation products, the disclosure also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the disclosure.

In some aspects, the protein of interest or polypeptide of interest is a recombinant protein that contains an Fc moiety and another domain, (e.g., an Fc-fusion protein). In some aspects, an Fc-fusion protein is a receptor Fc-fusion protein, which contains one or more extracellular domain(s) of a receptor coupled to an Fc moiety. In some aspects, the Fc moiety comprises a hinge region followed by a CH2 and CH3 domain of an IgG. In some aspects, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the II-1R1 extracellular region fused to Fc of hIgG1; see U.S. Pat. No. 6,927,004, which is herein incorporated by reference in its entirety), or a VEGF trap (e.g., aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other aspects, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

In one aspect, the protein of interest comprises a combination of any of the foregoing.

In some aspects, a sample can be prepared prior to or following enrichment steps, separation steps, and/or analysis steps. Preparation steps can include alkylation, reduction, denaturation, digestion, derivatization, and/or deglycosylation.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, the term “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl) phosphine hydrochloride (TCEP-HCl), or combinations thereof. A method of protein analysis, reduced peptide mapping, involves protein reduction prior to LC-MS analysis. In contrast, non-reduced peptide mapping omits the sample preparation step of reduction in order to preserve endogenous disulfide bonds. In some aspects, non-reduced preparation may be used, for example, in order to preserve an endogenous disulfide bond between Fab arms of an antibody or antibody-derived protein. In other aspects, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein or polypeptide. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein or polypeptide. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C), outer membrane protein T (OmpT), immunoglobulin-degrading enzyme of Streptococcus pyogenes (IdeS), IdeZ, igdE, glyserias, thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments. Journal Of Proteome Research, 2013, 12 1067-1077).

In some exemplary aspects, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, the term “deglycosylation” refers to refers to the removal of glycans from a glycan-containing molecule. Deglycosylation of a protein in a sample can be done enzymatically or chemically. In some aspects, deglycosylation of a protein in a sample comprises contacting the sample to PNGase F. The deglycosylation may comprise incubating the antibody preparation with PNGase F.

In some exemplary embodiments, methods for characterizing and/or quantifying a polypeptide or polypeptide multimer of interest can optionally comprise depleting an interfering molecule in the sample using immunoprecipitation (IP). As used herein, the term “immunoprecipitation” can include a process of precipitating a protein antigen out of solution using an antibody that specifically binds to that particular protein. Immunoprecipitation may be direct, in which antibodies for the target protein are immobilized on a solid-phase substrate, or indirect, in which free antibodies are added to the protein mixture and later captured with, for example, protein A/G beads. In some exemplary embodiments, IP may be conducted under native or near-native conditions, such that the native structure of a protein or proteins of interest are substantially preserved. The antibody preparation may be immuno-depleted prior to deglycosylation by incubating the antibody preparation with at least one immunoprecipitation antibody that binds to at least one molecule in the antibody preparation that is not the multimer of interest.

In some exemplary embodiments, the solid-phase substrate may be beads, for example agarose beads or magnetic beads. Beads may be coated in streptavidin in order to facilitate adherence to an antibody. A biotinylated “capture” antibody may then be contacted to the streptavidin-coated beads, adhering to the beads and forming “immunoprecipitation beads” capable of binding to the antigen of the adhered antibody.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC). In some aspects, a sample can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

In some exemplary aspects, the methods and systems of the present disclosure include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size. In some exemplary aspects, the mobile phase used to obtain an eluate from size exclusion chromatography can comprise a volatile salt. In some specific aspects, the mobile phase can comprise ammonium acetate, ammonium bicarbonate, or ammonium formate, or combinations thereof. In some specific aspects, the mobile phase can comprise ammonium acetate. In some exemplary aspects, the concentration of ammonium acetate is about 50-300 mM, about 100-200 mM, about 120-170 mM, or preferably about 150 mM. The methods may include a mobile phase for the size exclusion chromatography separation that comprises about 50 mM to about 300 mM ammonium acetate. In an exemplary embodiment, the concentration of ammonium acetate is about 50 mM, about 75 mM, about 100 mM, about 125 mM, about 150 mM, about 175 mM, about 200 mM, about 250 mM, or about 300 mM. In an exemplary embodiment, the concentration of ammonium acetate is 150 mM.

In some exemplary embodiments, the methods comprise identifying and/or quantifying a multimer of interest from an antibody preparation, the methods comprising: deglycosylating the antibody preparation to form a deglycosylated sample; separating the deglycosylated sample by size exclusion chromatography separation to form a separated sample; and analyzing the separated sample by nano-electrospray ionization (NSI) mass spectrometry analysis to identify and/or quantify the multimer of interest, wherein a mobile phase for the size exclusion chromatography separation that comprises 150 mM ammonium acetate.

Online coupling of SEC with direct MS detection under near native conditions (native SEC-MS) has gained interest in recent years to study mAb HMW species (Rouby, et al., supra; Ehkirch, A., Hernandez-Alba, O., Colas, O., Beck, A., Guillarme, D., Cianferani, S., Hyphenation of size exclusion chromatography to native ion mobility mass spectrometry for the analytical characterization of therapeutic antibodies and related products. J Chromatogr B Analyt Technol Biomed Life Sci, 2018, 1086, 176-183; Haberger, M., Leiss, M., Heidenreich, A. K., Pester, O., Hafenmair, G., Hook, M., Bonnington, L., Wegele, H., Haindl, M., Reusch, D. et al. Rapid characterization of biotherapeutic proteins by size-exclusion chromatography coupled to native mass spectrometry. MAbs, 2016, 8 (2), 331-339. Using MS-compatible mobile phases that can preserve protein conformation and non-covalent interactions, native SEC-MS (nSEC-MS) can provide rapid and improved identification of size variants or similarly sized polypeptides in a sample based on accurate mass measurement.

In some exemplary embodiments, size exclusion chromatography is a high-throughput size exclusion chromatography (HT-SEC). High-throughput size exclusion chromatography uses a shorter size exclusion chromatography column and a higher flow rate than size exclusion chromatography. In some aspects, the length of the HT-SEC column is 150 mm. The size exclusion chromatography may be high-throughput size exclusion chromatography and the separating can be performed with a 150 mm size exclusion chromatography column. In an exemplary embodiment, the high-throughput size exclusion chromatography column is 100 mm, 110 mm, 120 mm, 140 mm, 150 mm, 160 mm, 180 mm, or 200 mm in size.

In some aspects, the flow rate of HT-SEC is about 0.6 mL/min. The high-throughput size exclusion chromatography separation may be performed at a flow rate of about 0.3 to about 0.8 mL/min. The high-throughput size exclusion chromatography separation may be performed at a flow rate of about 0.3 mL/min, about 0.4 mL/min, about 0.5 mL/min, about 0.6 mL/min, about 0.7 mL/min, or about 0.8 mL/min. In an exemplary embodiment, the flow rate is about 0.6 mL/min.

In some exemplary embodiments, the methods comprise identifying and/or quantifying a multimer of interest from an antibody preparation, the methods comprising: deglycosylating the antibody preparation to form a deglycosylated sample; separating the deglycosylated sample by size exclusion chromatography separation to form a separated sample; and analyzing the separated sample by nano-electrospray ionization (NSI) mass spectrometry analysis to identify and/or quantify the multimer of interest, wherein the flow rate of the size exclusion chromatography column is 0.6 mL/min.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary aspects, the mass spectrometer may use a microflow nanospray ion source, for example Newomics® MnESI (microflow nanospray electrospray ionization) ion source, with M3 emitter. The ion source has multiple nozzles working together to split a single microflow stream evenly into multiple nanoflows. The MnESI source can be linked to a high-flow LC system using a T-splitter. This configuration allows a microflow to enter the MnESI, while the remaining analytical flow is directed towards an additional detector or system.

In some exemplary aspects, the mass spectrometer is a low-resolution mass spectrometer. In some aspects, the low-resolution mass spectrometer is configured to provide a mass resolution of 6,250 (at m/z=200). The mass spectrometry may be low-resolution mass spectrometry comprising a resolution setting of 6,250 (at m/z=200).

In some exemplary embodiments, the methods comprise identifying and/or quantifying a multimer of interest from an antibody preparation, the methods comprising: deglycosylating the antibody preparation to form a deglycosylated sample; separating the deglycosylated sample by size exclusion chromatography separation to form a separated sample; and analyzing the separated sample by nano-electrospray ionization (NSI) mass spectrometry analysis to identify and/or quantify the multimer of interest, wherein the mass spectrometry is low-resolution mass spectrometry comprising a resolution setting of 6,250 (at m/z=200).

In some aspects, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Picotti, P., and Aebersold, R., Selected reaction monitoring-based proteomics: workflows, potential, pitfalls and future directions. Nature Methods, 2012, 9, 555-566). MRM can be typically performed with triple quadrupole mass spectrometers wherein a precursor ion corresponding to the selected small molecules/peptides is selected in the first quadrupole and a fragment ion of the precursor ion is selected for monitoring in the third quadrupole (Choi, Y. S. et al., Targeted human cerebrospinal fluid proteomics for the validation of multiple Alzheimers disease biomarker candidates. Journal of Chromatography B, 2013, 930, 129-135).

In some aspects, LC-MS can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In some aspects, native MS allows for better spatial resolution compared to non-native MS. For detailed review on native MS, refer to the review: Erba, E. B., and Pe-tosa, C., The emerging role of native mass spectrometry in characterizing the structure and dynamics of macromolecular complexes. Protein Science, 2015, 24, 1176-1192.

The present disclosure will be more fully understood by reference to the following examples. They should not, however, be construed as limiting the scope of the disclosure.

### EXAMPLES

Chemicals and reagents. All mAbs (Ab1-Ab10), anti-drug antibody (against Ab9), and fixed dose co-formulation (FDC) products were produced at Regeneron Pharmaceuticals, Inc. (Tarrytown, NY). Ammonium acetate and dimethyl sulfoxide (DMSO) were purchased from Sigma-Aldrich (St. Louis, MO). EZ-Link™ NHS-Biotin, Pierce™ High Capacity Streptavidin Agarose, Pierce™ micro-spin columns, formic acid (FA), and UltraPure 1 M Tris-HCl buffer, pH 7.5 were purchased from Thermo Fisher Scientific (Waltham, MA). PNGase F was purchased from New England Biolabs (Ipswich, MA).

Deglycosylation of co-formulated antibodies (Abs). Deglycosylation treatment was performed by mixing each mAb cocktail sample with PNGase F (1 IUB milliunit per 10 μg of protein) and incubating at 45° C. for 1 h in 50 mM Tris-HCl buffer (pH 7.5) to remove the Fc N-glycans. After the deglycosylation treatment, samples were centrifuged at 14,000 g for 5 min. The supernatants were then transferred to HPLC vials and ready for native SEC-MS analysis.

Native SEC-UV-MS analysis. Native SEC chromatography was performed on an UltiMate 3000 UPLC (Thermo Fisher Scientific, Bremen, Germany) equipped with a Waters Acquity BEH protein SEC column (4.6×300 mm, 1.7 μm, 200 Å; Milford, MA) with the column compartment set to 30° C. An injection amount of 6 to 20 μg of the mAb cocktail samples was loaded on column for each analysis. SEC separation was achieved using an isocratic gradient of mobile phase A containing 150 mM ammonium acetate (pH 6.8) operated at a flow rate of 0.2 mL/min. The post-column flow was split into a microflow (<10 μL/min) for nano-electrospray ionization MS detection and a remaining high flow for UV detection (280 nm). A Thermo Q-Exactive UHMR mass spectrometer (Thermo Fisher Scientific, Bremen, Germany) equipped with a microflow-nanospray electrospray ionization (MnESI) source and a M3 multinozzle emitter (Newomics, Berkeley, CA) was used for MS data acquisition. MS resolution settings of 12,500 or 6,250 (at m/z=200) were used for data acquisition. The scheme of the nSEC-UV/MS platform interface is depicted in FIGS. 1 and 2. Further details on the experimental setup and instrument parameters can be found in a previous publication (Yan, Y. et al., Versatile, Sensitive, and Robust Native LC-MS Platform for Intact Mass Analysis of Protein Drugs. J Am Soc Mass Spectrom., 2020, 31 (10), 2171-2179).

For the high-throughput SEC-MS (HT-SEC-MS) method, a 150 mm Acquity BEH protein SEC column (4.6× 150 mm, 1.7 μm, 200 Å; Milford, MA) was used with an increased mobile phase flow rate at 0.6 mL/min for mAb size variant separation. All other operation parameters were kept the same as SEC-MS methods described above.

Immunodepletion using anti-drug antibody for the three-mAb cocktail sample consisting of Ab8, Ab9, and Ab10. The anti-Ab9 Ab sample was biotinylated by mixing the 10 mM NHS-biotin solution and the 10 mg/mL anti-Ab9 Ab solution at 12:1 molar ratio followed by incubating at room temperature for 30 min. The reaction mixture was subsequently buffer exchanged into 100 mM Tris-HCl (pH 7.5) solution using an Amicon 30 kDa MWCO ultra centrifugal filter (MilliporeSigma, Burlington, MA). The final protein concentration of biotinylated anti-Ab9 Ab was determined by Nanodrop 2000 spectrophotometer (ThermoFisher Scientific, Waltham, MA USA) at 280 nm. Using a published protocol, 2.5 nmole of the biotinylated anti-Ab9 Ab in 50 μL 100 mM Tris-HCl was added to 25 μL of high capacity streptavidin resin (equivalent to 50 μL of resin slurry) in an empty microspin column to prepare immobilized anti-Ab9 Ab (Zhang, Z. et al., A competitive binding-mass spectrometry strategy for high-throughput evaluation of potential critical quality attributes of therapeutic monoclonal antibodies. MAbs., 2022, 14 (1), 2133674). The microspin column containing immobilized anti-Ab9 Ab was then added with 20 μL of the cocktail sample composed of Ab8, Ab9, and Ab10 (10 mg/mL) and incubated at room temperature for 45 min. Following incubation, the supernatant (flow-through) fraction was collected by centrifuging the microspin column at 3000 g for 1 min. The collected flow-through sample, together with one aliquot of the untreated cocktail sample, were then subjected to native deglycosylation followed by nSEC-MS analysis.

Data analysis. The UV peak area was integrated using Xcalibur software (Thermo Fisher Scientific, Waltham, MA USA). Deconvoluted MS spectra were generated using IntactMass™ software (Protein Metrics, Boston, MA USA). To calculate the total dimer abundance in each sample, the UV-based peak area of the dimer was compared to the combined peak areas of both dimer and monomer peaks. The relative abundance (distribution) of each dimer species within the total dimer peak of a mAb cocktail sample was calculated using its integrated mass peak area and compared against the summed mass peak areas of all dimers. Lastly, the absolute abundance of each dimer in the cocktail sample was calculated by multiplying the UV-based total dimer abundance with the MS-determined relative abundance of each dimer.

### Example 1. Quantitative Performance of nSEC-MS/UV Method Using a MS-Compatible Mobile Phase

SEC separates biomolecules based on their hydrodynamic radii and, when combined with UV detection, is widely used as a quality control (QC) method for monitoring HMW contents in therapeutic mAbs. A SEC method is often operated using mobile phases containing high concentrations of nonvolatile salts, such as sodium chloride or sodium perchlorate, to minimize the undesired protein-matrix interactions (e.g., electrostatic interaction) and hence achieve good quantitative recovery. Recently, online native MS detection coupled with SEC-UV (nSEC-UV/MS) analysis has emerged as a valuable tool for therapeutic mAb characterization by allowing sensitive and unambiguous identification of low-abundance size variants. In particular, with additional separation in the mass dimension, co-eluting dimers from the co-formulated mAb cocktail samples could potentially be differentiated and individually quantitated. However, as online MS detection requires the use of volatile salts in the SEC mobile phase (e.g., ammonium acetate), which may lead to different protein retention and recovery behavior compared to the SEC buffers, its quantitative performance should be thoroughly evaluated.

To facilitate nSEC-UV/MS analysis, a previously developed platform was adopted, which uses an analytical-scale SEC column, an ammonium acetate-based mobile phase (150 mM, pH 6.8), and nanospray ionization (NSI) for sensitive MS detection (FIG. 2, Yan, Y. et al., 2013, Yan, Y. et al., 2020). Notably, the employment of a relatively high concentration of ammonium acetate in the mobile phase was crucial to obtain good protein recovery from SEC analysis, as a lower concentration may lead to increased electrostatic interaction between mAb molecules and the column matrix (Yan, Y. et al., Coupling Mixed-Mode Size Exclusion Chromatography with Native Mass Spectrometry for Sensitive Detection and Quantitation of Homodimer Impurities in Bispecific IgG. Anal Chem., 2019, 91 (17), 11417-11424). Subsequently, the adoption of NSI for MS analysis was essential to mitigate the ion suppression effect from ammonium salt and achieve sensitive MS detection.

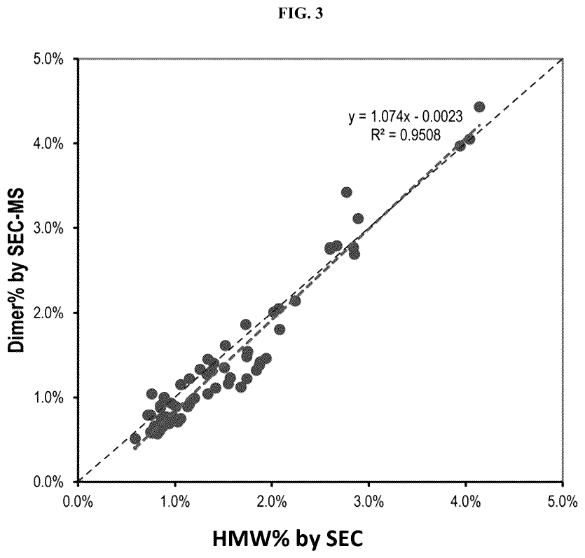

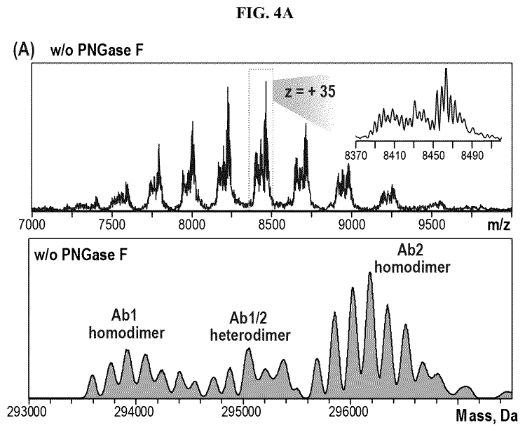

Using 80 mAb cocktail samples as test subjects, the quantitation results of the dimers (based on UV peak measurement) generated by the nSEC-UV/MS method using 150 mM ammonium acetate as mobile phase were compared to those obtained by a SEC-UV method used in quality control (mobile phase: 0.5-1 M of sodium chloride or sodium perchlorate, per molecule optimization). As shown in FIG. 3, the relative abundances of the dimer (dimer %) obtained by the two methods exhibited a linear correlation, with the coefficient of determination (R2) and slope determined to be 0.9508 and 1.074, respectively. These results suggest that across a large set of mAb samples, the developed nSEC-UV/MS method, despite using a different mobile phase, achieved good quantitative consistency with SEC-UV methods. It was therefore concluded that the nSEC-UV/MS method may be reliably implemented for dimer detection and quantitation during mAb cocktail development, maintaining good alignment with results from SEC-UV methods that are widely used in process/formulation development and in quality control.

### Example 2. Native Deglycosylation Improves Heterodimer Analysis by nSEC-MS

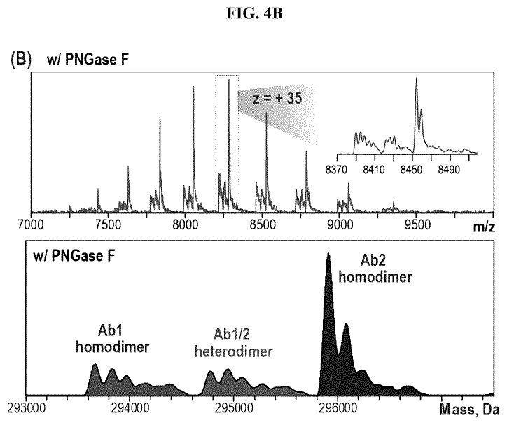

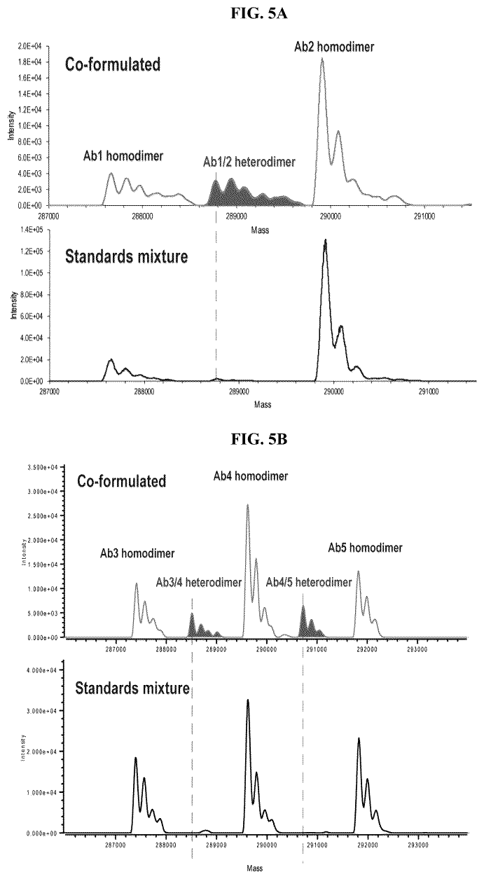

Both IgG1 and IgG4, the most commonly used subclasses in therapeutic mAbs, are decorated with N-linked glycans at the conserved N-glycosylation site N297 (EU numbering system) in the heavy chain CH2 domain. This N-glycosylation can exhibit both macro—(i.e., the N-glycan occupancy) and micro-heterogeneities (i.e., the N-glycan structure), which lead to undesired mass heterogeneity of mAbs during MS analysis, resulting in increased spectral complexity and decreased detection sensitivity. This mass heterogeneity could be particularly problematic for analyzing dimer species in mAb cocktail samples, where the different hetero- and homodimers are not only present at low abundances but also often exhibit close molecular weights that can overlap with each other. To demonstrate the impact of N-glycosylation, a two-mAb fixed-dose co-formulation (FDC) sample (consisting of Ab1 and Ab2) that was stored at 5° C. for 3 months was subjected to nSEC-MS analysis to obtain the mass spectra of the dimers (FIG. 4A). Based on the raw mass spectra and the deconvoluted mass spectra, three different dimer populations were detected, corresponding to Ab1 homodimer, Ab1/2 heterodimer, and Ab2 homodimer respectively, which were consistent with their predicted masses (Table 1). However, due to the mass heterogeneity originated from the N-linked glycans, each dimer exhibited a broad cluster of mass peaks, leading to notable mass overlaps with each other. In particular, as the heterodimer exhibits a molecular weight that falls right between the two homodimers, its mass profile could not be clearly resolved from either homodimer, rendering MS-based quantitation challenging.

TABLE 1
Predicted masses of Ab dimers.

| | Predicted mass (Da, w/o | Predicted mass (Da, w/ |
|---|---|---|
| Dimer | glycan) | glycan*) |
| Ab1 homodimer | 287638.6 | 293416.0 |
| Ab2 homodimer | 289889.8 | 295667.2 |
| Ab3 homodimer | 287383.0 | |
| Ab4 homodimer | 289613.6 | |
| Ab5 homodimer | 291815.8 | |
| Ab6 homodimer | 287138.2 | |
| Ab7 homodimer | 290165.8 | |
| Ab8 homodimer | 287558.6 | |
| Ab9 homodimer | 288267.0 | |
| Ab10 homodimer | 290249.2 | |
| Ab1/2 heterodimer | 288764.2 | |
| Ab3/4 heterodimer | 288498.3 | |
| Ab3/5 heterodimer | 289599.4 | |
| Ab4/5 heterodimer | 290714.7 | |
| Ab6/7 heterodimer | 288652.0 | |
| Ab8/9 heterodimer | 287912.8 | |
| Ab8/10 heterodimer | 288903.9 | |
| Ab9/10 heterodimer | 289258.1 | |

*The predicted masses with glycan are based on G0F on both arms of antibody.

To tackle this challenge, PNGase F-mediated deglycosylation treatment was explored with the goal of removing the Fc N-glycans and converting various mAb glycoforms into a single deglycosylated population. To preserve the non-covalent dimers that may be present in the sample, the deglycosylation reaction was conducted under mild conditions without any denaturants or surfactants (45° C. for 1 h, pH 7.5). With deglycosylation treatment, all three dimer species exhibited distinct mass profiles that were sufficiently resolved from each other, allowing for unambiguous mass peak integration for individual dimer quantitation (FIG. 4B). In addition to enhanced mass separation, a concomitant improvement in MS intensity, signal-to-noise ratio (S/N), and spectral quality was observed for the deglycosylated sample, presumably due to the signal consolidation from various mAb glycoforms, as a result of N-glycan removal. This further improved the method sensitivity, which was particularly critical for samples containing very low levels of dimers.

Although the deglycosylation reaction was carried out at elevated temperature, the applicants hypothesized that artificial heterodimer formation during this treatment should be negligible, considering the short incubation time (i.e., 1 hour) as well as the low mAb concentrations (<5 mg/mL) used in this reaction. To evaluate the impact of this treatment on artificial heterodimer formation, the individual Ab1 and Ab2 formulated drug substance (FDS) samples were mixed shortly before the deglycosylation treatment and then analyzed by nSEC-MS analysis. No apparent mass peak corresponding to the heterodimer was observed from the freshly prepared Ab1 and Ab2 mixture sample, suggesting the selected deglycosylation conditions were mild enough to prevent artificial dimer formation (FIG. 5A). Meanwhile, as a positive control experiment, the aforementioned FDC sample of Ab1 and Ab2 (5° C. for 3 months) was also analyzed in parallel, which revealed the successful detection of the heterodimer species. Further, a similar study using a three-mAb FDC sample was also conducted and achieved the same conclusion (FIG. 5B). Specifically, the individually formulated Ab3, Ab4, and Ab5 FDS samples were freshly mixed at a 1:1:1 ratio and compared against the corresponding FDC sample for dimer detection after deglycosylation treatment. Consistently, heterodimers were only detected in the FDC sample but absent in the freshly mixed sample, further supporting the conclusion that the applied deglycosylation treatment did not introduce artificial dimers. Since the native deglycosylation treatment did not impact heterodimer quantitation, it was implemented in all subsequent dimer analyses.

### Example 3. Characterization of Heterodimer Growth in FDC Stability Studies by nSEC-MS

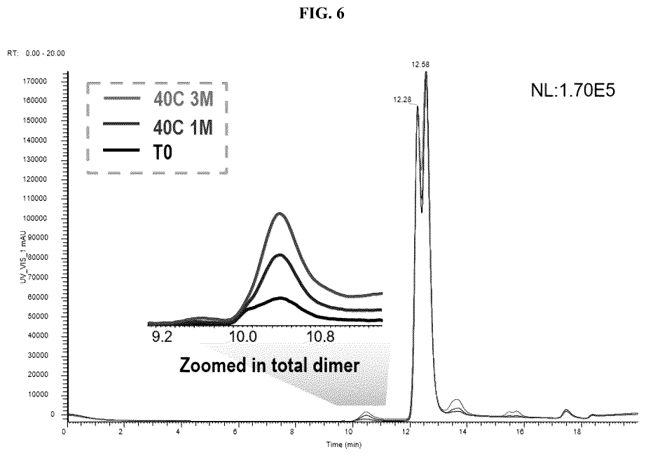

To demonstrate its applicability in mAb FDC development, the nSEC-UV/MS method was implemented to monitor the heterodimer growth during the stability studies of an FDC sample. The FDC sample, which consisted of 1:1 mixed Ab1 and Ab2, was placed under different storage/thermal stress conditions (5° C., 25° C., and 40° C.) for extended periods of time (up to 6 months). Aliquots were subsequently removed at different time points and subjected to native deglycosylation followed by nSEC-UV/MS analysis. As shown in FIG. 6, the increasing presence of heterodimers in the FDC sample at 40° C. could be monitored by UV absorbance over time. For quantitation of individual dimers, the integrated peak area of each dimer from the deconvoluted mass spectra was used to estimate its relative abundance within the dimer population (FIG. 6 insets). When combined with the UV-measured total dimer percentage, this approach can report the abundance of each individual hetero- or homodimer in the sample.

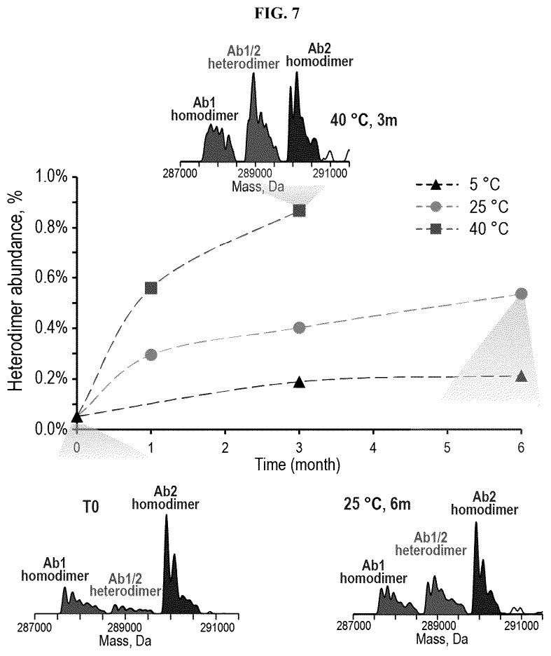

Using this strategy, the abundance of heterodimer from each sample was quantitated, and its growth over time was plotted under different thermal conditions (FIG. 7). The heterodimer abundance increased over the course of the stability studies. Additionally, a positive correlation between the heterodimer growth rate and the storage/stress temperature was observed, showing the fastest growth at 40° C. (an average of 0.27% per month), a moderate growth at 25° C. (an average of 0.08% per month), and the slowest growth at the 5° C. storage conditions (an average of 0.03% per month). Interestingly, by comparing the relative distribution of hetero- and homodimers in the deconvoluted mass spectra, it was apparent that the relative abundances of heterodimer were significantly higher in the stressed samples (e.g., 25° C. at 6 months and 40° C. at 3 months) than in the control sample (TO) (FIG. 6 insets). This suggested a higher propensity of heterodimer formation than homodimer formation under thermal stress conditions. These findings highlight the importance of monitoring heterodimer formation during mAb cocktail co-formulation development and nSEC-UV/MS as a valuable tool for studying this critical quality attribute.

### Example 4. Analysis of Heterodimers by High-Throughput nSEC-MS (HT-SEC-MS)

Formulation development for mAb cocktail drug products is an intricate process and often involves comprehensive evaluation of the relationships between the formulation components (e.g., buffer, pH, excipients, etc.) and the product attributes through various stability studies (e.g., different temperatures, low/high pH, freeze-thaw, light exposure, agitation, etc.). This often generates a large number of samples that can be benefited from high-throughput analytical methods. For nSEC-MS-based heterodimer analysis, although the deglycosylation reaction can be automated and performed in a multiplexed fashion, the nSEC-MS analysis remains a speed-limiting step, taking 20 minutes per sample.

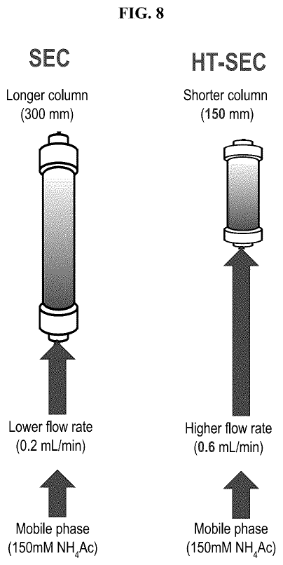

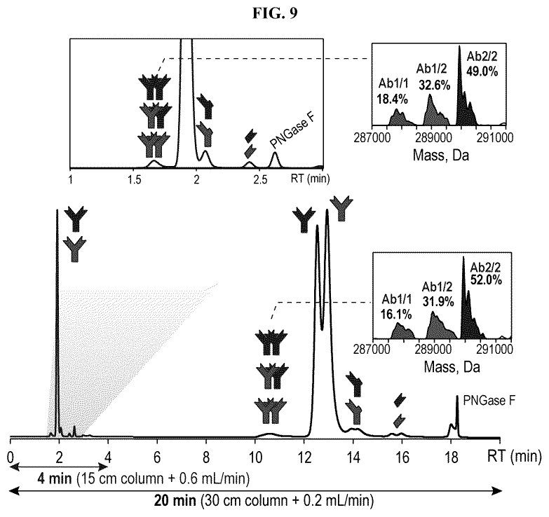

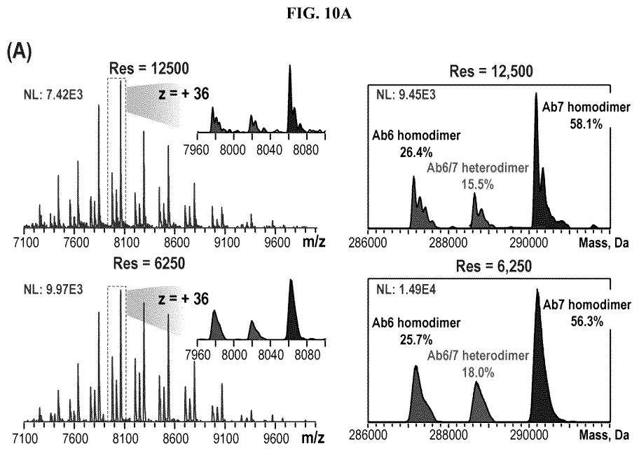

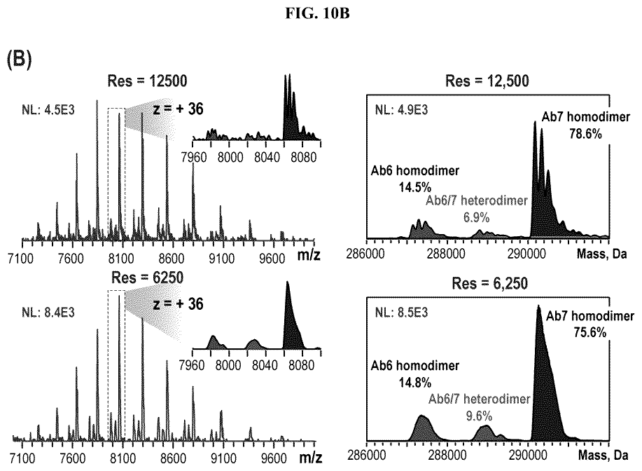

In order to reduce the nSEC-MS analysis time, the feasibility of using a shorter SEC column (15 cm vs 30 cm) and a higher flow rate (0.6 mL/min vs 0.2 mL/min) was explored (FIG. 8). Notably, the adoption of the previously established nLC-MS platform, which utilizes a split-flow design and a multi-nozzle emitter, was key to accommodating this increased flow rate and providing sensitive MS measurements (Yan, Y. et al., 2020). Using a stressed FDC sample composed of Ab1 and Ab2 (25° C., 20 months), the performance of this high-throughput nSEC-MS method (HT-SEC-MS) was evaluated and compared with the nSEC-MS method. As shown in FIG. 9, analysis of the FDC sample by the HT-SEC-MS method was completed in just 3 minutes, which represented a 5-fold improvement in throughput compared to the nSEC-MS method (20 min). Further, the two methods demonstrated overall similar separation performance, consistently resolving four groups of UV peaks that corresponded to the dimeric, monomeric, and low molecular weight (LMW) species of Ab1 and Ab2, respectively. The detected LMW species were identified as a truncated mAb species (i.e., mAb losing an Fab arm and the Fab fragment) due to the protein backbone clippings at the upper hinge region, which is common for IgG1 molecules under prolonged thermal stress conditions (25° C., 20 months).

Most importantly, the HT-SEC-MS method also demonstrated baseline separation of the dimer and the monomer, which was essential for obtaining accurate UV-based quantitation of the total dimer and achieving sensitive MS measurements. Interestingly, the nSEC-MS method also displayed partial separation of the Ab1- and Ab2-related size variants (e.g., split peaks for dimer, monomer, and LMWs), likely driven by the different secondary interactions between the mAb molecules and the column matrix due to their distinct surface properties. In contrast, this molecule-specific separation was not observed in the HT-SEC-MS method, suggesting less prominent protein-matrix interactions under the HT-SEC-MS conditions. The reduced secondary interaction from the HT-SEC-MS method is presumably attributed to the decreased surface areas of the column matrix due to the use of a shorter column and the increased mobile phase-to-stationary phase volume ratio due to the use of a higher flow rate. It is worth noting that eluting different dimer species within a single confined UV peak is beneficial for quantitative analysis. It not only simplifies the process of integrating all mass spectra across the entire dimer elution peak for deconvolution, but also reduces the ionization discrepancy, as the various hetero- and homodimer species are all ionized under identical solvent/MS conditions.

In addition to chromatography resolution, quantitative performance between the nSEC-MS and HT-SEC-MS methods was also evaluated. To this end, both the UV-based quantitation of the total dimer and the MS-based quantitation of the dimer distribution within the dimer peak were obtained and compared between the two methods. Specifically, using the UV peak areas, the total dimer abundances (dimer %) from both SEC-UV analyses of the FDC sample were found to be identical at 1.8%. Meanwhile, using the integrated MS peak areas from the deconvoluted mass spectra of the dimer peak, the relative distributions of Ab1 homodimer, Ab1/2 heterodimer, and Ab2 homodimer were determined to be highly comparable between the HT-SEC-MS and the nSEC-MS methods (18.4%-32.6%-49.0% vs 16.1%-31.9%-52.0%) (FIG. 9). Together, these results demonstrate that the HT-SEC-MS method has comparable quantitative performance to nSEC-MS, highlighting its potential to achieve both accurate and high-throughput quantitation of heterodimers to support mAb cocktail co-formulation studies.

### Example 5. Analysis of Heterodimers by Low-Resolution nSEC-MS (LR-SEC-MS)

As heterodimers can only be formed after the mixing of individual mAb samples, their relative abundances are usually low in the FDC samples under normal storage or mild stress conditions. In addition, the absolute abundances of the heterodimers are also highly dependent on the concentrations of the individual mAbs. For example, intravenous (IV) compatibility studies are often required during formulation development to support mAb cocktail co-administration. In such studies, individual mAbs are often diluted and mixed at low concentration ranges (e.g., <1 mg/mL), which typically leads to very low levels of heterodimers and poses significant challenges for MS detection (Riccardi, C. et al., J Pharm Sci., 2023, 112(12), 3045-3055, and Joubert, M. K. et al., Classification and characterization of therapeutic antibody aggregates. J Biol Chem., 2011, 286(28), 25118-33). Therefore, strategies that can effectively improve the MS detection sensitivity are highly desirable to achieve successful detection and quantitation of the low-abundance heterodimers, particularly for the unstressed FDC samples and IV compatibility samples.

To improve the MS detection of dimer species, a strategy known as “proteoform integration”, which was first reported by Nagornov et al., was explored to enhance the drug-to-antibody ratio (DAR) measurement of antibody-oligonucleotide conjugates (Nagornov, K. O. et al., Drug-to-Antibody Ratio Estimation via Proteoform Peak Integration in the Analysis of Antibody-Oligonucleotide Conjugates with Orbitrap Fourier Transform Mass Spectrometry. Anal Chem., 2021, 93 (38), 12930-12937). In this strategy, the Orbitrap-based MS data acquisition was conducted under very low resolution settings so that the related proteoforms from a single DAR species were consolidated, leading to significantly simplified spectra and an improved signal-to-noise ratio (S/N).

The mAb dimer species, although having undergone deglycosylation, still existed in multiple proteoforms due to the presence of various post-translational modifications (PTMs). Therefore, the applicants hypothesized that a similar strategy may also benefit the MS detection of various hetero- and homodimer species present in a mAb cocktail sample. It is worth noting that the applicants previously demonstrated that a mass resolution setting of 12,500 (at m/z=200) was appropriate for typical native LC-MS analysis of mAbs, as it allowed for baseline resolution of common glycoforms (Δmass=162 Da) for mAb monomer while providing excellent sensitivity for mAb variants (e.g., ˜ 0.1% for mAb dimer) (Yan, Y. et al., 2020).

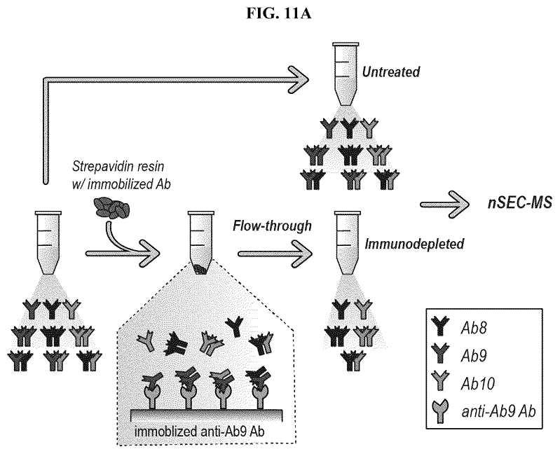

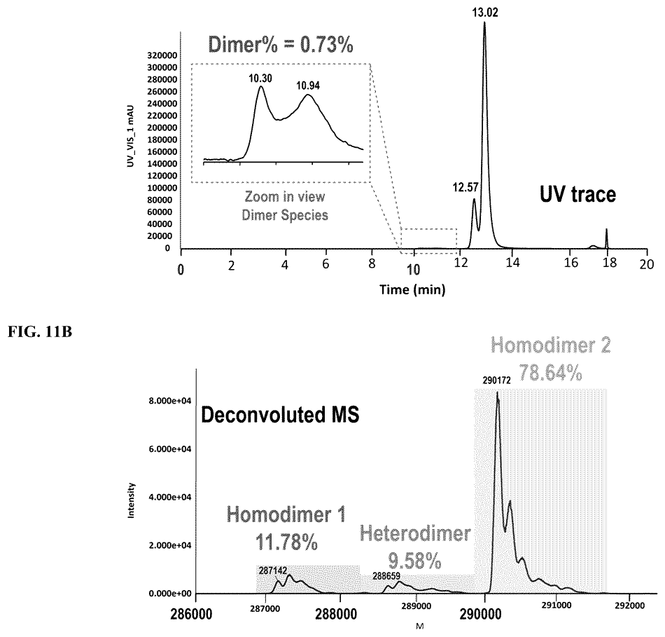

To determine if a lower resolution setting can further improve the MS detection of dimer species, nSEC-MS analyses under two resolution settings (R=6,250 and R=12,500) were compared using a two-mAb cocktail sample. This mAb cocktail sample was co-formulated with 40 mg/mL Ab6 and 50 mg/mL Ab7 and stored at −80° C. prior to the analysis. As determined by SEC-UV measurement, the total dimer abundance in this sample was estimated at 0.66%, which was further characterized by MS to consist of three dimers: Ab6 homodimer, Ab6/7 heterodimer, and Ab7 homodimer. From the native mass spectra, it was clear that both MS resolution settings led to the unambiguous detection and differentiation of the three dimers (FIG. 10A, left panels). Under the resolution setting of 12,500, each dimer exhibited multiple peak features that likely corresponded to different dimer proteoforms, such as the unmodified form and those with various PTMs (e.g., unprocessed C-terminal Lys and Lys glycation). These peak features may be valuable to understand the heterogeneity of each dimer species; however, they did not facilitate the dimer distribution measurement. In contrast, these peak features were not observed in the mass spectrum acquired under the low resolution setting of 6,250. Instead, for each charge state, a single and unresolved m/z peak was observed for each dimer species (FIG. 10A, insets).

Considering that both resolution settings produced well-resolved mass spectra, MS-based relative quantitation of these three dimers should not be affected under these two conditions. Indeed, using the integrated mass peak areas from the deconvoluted mass spectra, the dimer distributions were measured similarly at R=12,500 (26.4%, 15.5%, and 58.1%, respectively) and at R=6,250 (25.7%, 18.0%, and 56.3%, respectively) (FIG. 10A, right panels). Importantly, a concomitant improvement in signal intensity and S/N ratio was observed for both the raw mass spectrum and the deconvoluted mass spectrum acquired at R=6,250 compared to those at R=12,500, highlighting the benefit of enhanced MS detection through dimer proteoform integration.

This dimer proteoform integration strategy, as achieved by the LR-SEC-MS method, is particularly valuable for analyzing low-abundance dimer species in mAb cocktail samples. To showcase its importance in improving dimer detection and quantitation, this method was subsequently utilized to study the heterodimer formation in an IV compatibility sample consisting of Ab6 and Ab7. To mimic the co-administration process, this sample was prepared by mixing individual Ab6 and Ab7 drug products in IV diluent at concentrations of 1 mg/mL and 5 mg/mL, respectively, stored at 5° C. for 24 hours, and then kept at room temperature overnight. Using SEC-UV analysis, the total dimer abundance in this sample was measured to be 0.36%. However, only a very small fraction was anticipated to be contributed by Ab6/7 heterodimer, due to the lower concentration of Ab6. Indeed, native mass spectra of the dimer peak revealed a predominant population of Ab7 homodimer (>75% relative abundance) and much lower relative abundances of the other two dimers (FIG. 10B). Notably, at R=12,500, both Ab6 homodimer and Ab6/7 heterodimer exhibited a poor S/N ratio for each of their charge states in the raw mass spectrum. This is problematic as it may lead to under-representation of the corresponding species in the deconvoluted mass spectra, thus compromising the accuracy of MS-based quantitation (FIG. 9B, top panel).

In contrast, through dimer proteoform integration, the LR-SEC-MS method (R=6,250) led to a significant improvement in both overall signal intensity and S/N ratio for all three dimer species, and allowed for confident spectral deconvolution (FIG. 10B, bottom panel). As a result, more reliable quantitation of the dimer distribution can be achieved. In this example, the relative abundance of Ab6/7 heterodimer within the total dimer peak was measured at 9.6% at R=6,250. However, it was likely underestimated at 6.9% at R=12,500 due to poor detectability. Considering the total dimer was only present at 0.36% in this sample, the absolute abundance of this heterodimer was estimated to be only 0.035% (or 3.5 ng based on an injection amount of 10 μg), highlighting the excellent dynamic range/sensitivity achieved by this method.

### Example 6. Differentiation and Quantification of Dimer Species in Three-mAb Cocktails by Immunodepletion-Assisted nSEC-MS (ID-SEC-MS)

To achieve the desired therapeutic outcome, antibody cocktail products sometimes contain more than two mAbs. For instance, REGN-EB3 (Inmazeb), the first Food and Drug Administration (FDA) approved therapeutic for the treatment of EBOV infection, is composed of three different mAbs each targeting different epitopes of the virus. In such a three-mAb system, heterodimer characterization becomes more challenging due to the increased complexity arising from the many possible dimer species present in the mixture samples (e.g., either during co-formulation development or during IV compatibility assessment for co-administration). For example, while a two-mAb cocktail sample may contain up to three dimer species (i.e., two homodimers and one heterodimer), a three-mAb cocktail sample could contain a total of six different dimer species (i.e., three homodimers and three heterodimers). The increased number of dimer species poses a significant challenge for their differentiation by mass-based measurement. This is particularly true considering the often similar molecular weights of mAb molecules (i.e., ˜150,000 Da) and the broad mass profiles of each dimer (i.e., 500-1,000 Da) due to the presence of multiple proteoforms. In many cases, two or more dimer species within the same mixture sample may exhibit overlapping mass profiles, rendering MS-based differentiation and quantitation impossible.

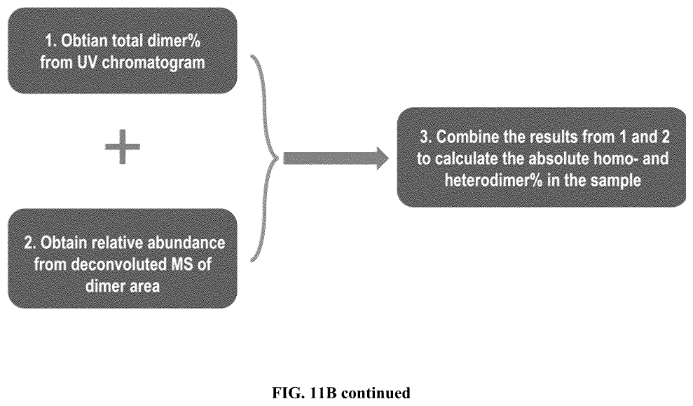

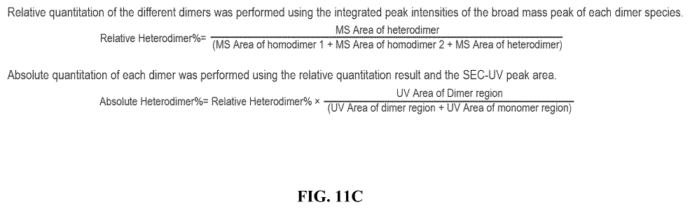

To cope with the challenges associated with dimer analysis in three-mAb cocktail samples, an immunodepletion-assisted SEC-MS (ID-SEC-MS) workflow was developed (FIG. 11A). Specifically, by using resins coated with an anti-drug Ab that specifically binds to one of the mAb components in the cocktail, multiple dimer species containing this mAb can be removed. This treatment leads to greatly simplified dimer complexity, thereby facilitating MS-based dimer differentiation and quantitation. Finally, combined with the results from the untreated sample, the abundances of the removed dimer species can also be back-calculated.

An overall strategy for dimer quantification after immunodepletion is illustrated in FIG. 11B. The nSEC-UV chromatogram is used to determine a percent of total dimers in the sample, and the deconvoluted mass spectra is used to determine a relative abundance of each dimer species. The information from the nSEC-UV chromatogram and deconvoluted mass spectrum is then combined to calculate the percent of each dimer species in the sample, including homo- and heterodimers. The calculations for the relative quantitation of dimer species using data from the nSEC-UV chromatogram and the deconvoluted mass spectra are illustrated in FIG. 11C.

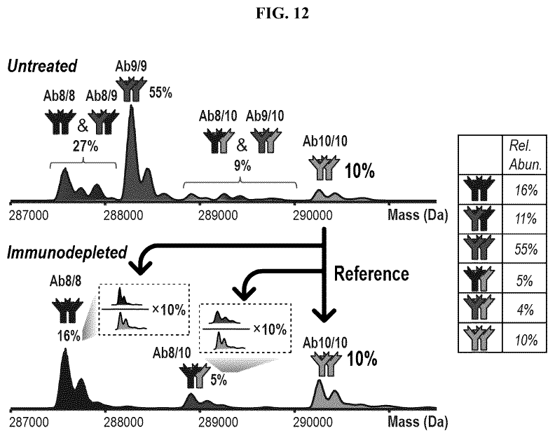

To illustrate this workflow, a three-mAb cocktail sample (60 mg/mL Ab8, 60 mg/mL Ab9, and 60 mg/mL Ab10, stored at 25° C. for 1 month) was used as an example. As shown in the dimer mass spectrum, due to mass profile overlaps between the dimer species, the six possible dimer species were resolved into four distinct mass groups for relative distribution quantitation, including 1) Ab8 homodimer and Ab8/9 heterodimer (27%), 2) Ab9 homodimer (55%), 3) Ab8/10 heterodimer and Ab9/10 heterodimer (9%), and 4) Ab10 homodimer (10%) (FIG. 12, top panel).

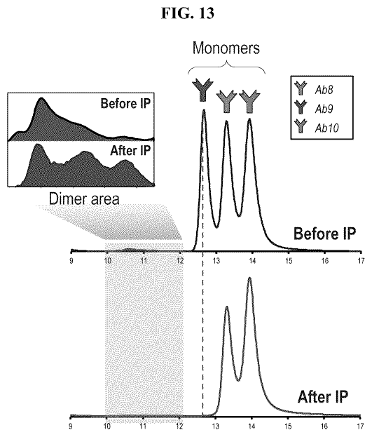

To assess the individual dimer contribution in the two mass groups (Group 1 and 3) that contain two dimer species, immunodepletion using immobilized anti-Ab9 antibody (A9A) was applied to the cocktail sample. This treatment should remove all the Ab9-containing species, including Ab8/9 heterodimer in Group 1 and Ab9/10 heterodimer in Group 3, thereby eliminating their interference and allowing for accurate quantitation of the remaining dimer species (i.e., Ab8 homodimer in Group 1 and Ab8-Ab10 heterodimer in Group 3). The nSEC-UV/MS analysis demonstrated complete removal of Ab9 monomer from the immunodepleted cocktail sample (FIG. 13). Additionally, the UV peaks corresponding to the dimer species also displayed a notable change in profile as well as a decrease in abundance after the immunodepletion treatment, presumably resulting from the removal of Ab9-containing dimers. This was subsequently confirmed by MS analysis of the dimer peak, which showed the complete removal of Ab8/9 heterodimer, Ab9 homodimer, and Ab9/10 heterodimer, resulting in a greatly simplified spectrum, with the remaining three dimer species (i.e., Ab8 homodimer, Ab8/10 heterodimer, and Ab10 homodimer) being well resolved from each other (FIG. 12, bottom panel).

Using the mass peak areas, the abundance ratios between these three dimer species were readily established. As Ab10 homodimer (10%) can be reliably quantitated in the untreated sample free from interference, it can be used as a reference to deduce the abundances of the other two dimers. Specifically, using the measured ratios of [Ab8 homodimer]-to-[Ab10 homodimer] and [Ab8/10 heterodimer]-to-[Ab10 homodimer] from the immunodepleted cocktail sample, the relative abundances of Ab8 homodimer and Ab8/10 heterodimer in the untreated cocktail sample were deduced to be 16% and 5%, respectively (FIGS. 11C and 12). Subsequently, through simple subtraction, the relative abundances of Ab8/9 heterodimer and Ab9/10 heterodimer were back-calculated to be 11% (i.e., 27%-16%) and 4% (9%-5%), respectively. Collectively, the individual dimer abundances of all six dimer species in this three-mAb cocktail sample were determined by this immunodepletion-assisted nSEC-MS (ID-SEC-MS) method. Lastly, this ID-SEC-MS method may also be applicable to mAb cocktails comprising of more than three mAbs. However, multiple immunodepletion treatments may be necessary to tease out the individual dimer contributions among the complex dimer mixture.

Antibody cocktails can offer some unique therapeutic advantages over antibody monotherapies; however, they also present unusual analytical challenges. Heterodimers, formed via inter-molecular interactions between two different mAbs upon co-formulation or co-administration, are important quality attributes to monitor during the development of mAb cocktails. In this study, the applicants report the development and evaluation of a widely applicable nSEC-MS method for highly effective dimer analysis in mAb cocktails. Different sample treatment strategies were developed to facilitate dimer differentiation and quantitation in both two-mAb and three-mAb cocktails. In addition, key parameters in SEC separation and MS data acquisition were explored for improved method sensitivity and throughput.

First, the quantitative performance of the nSEC-UV/MS method using an MS-compatible mobile phase (150 mM ammonium acetate) was evaluated and demonstrated highly comparable to those using SEC mobile phases. Secondly, a mild deglycosylation step was assessed and found to significantly improve the differentiation and quantitation of hetero- and homodimers by reducing the mass heterogeneity from the Fc N-glycans. Meanwhile, this treatment was demonstrated to not cause artifactual dimer formation, thereby maintaining the quantitation accuracy. This workflow was then applied to investigate the heterodimer growth in a two-mAb cocktail under different storage/thermal stress conditions. The study showed a direct correlation between heterodimer growth rate and storage/thermal stress temperature. Further, to accommodate the large sample size from mAb co-formulation studies, an HT-SEC-MS method was developed, which achieved a 5-fold improvement in method throughput compared to the method while maintaining similar quantitative performance. Additionally, a dimer proteoform integration strategy, achieved by low-resolution MS data acquisition, was demonstrated to be highly effective in enhancing the MS detection of mAb dimers. Lastly, to cope with the increasingly complex dimer mixtures, such as those present in three-mAb cocktails, an immunodepletion-assisted strategy was developed. This method was applied to a three-mAb cocktail sample and achieved successful relative quantitation of all six dimer species. Together, the applicants demonstrate that with the developed nSEC-MS method and the associated analytical strategies, identification and quantitation of hetero- and homodimers from mAb cocktails can be achieved with high sensitivity and method throughput. To the best of our knowledge, this method represents the first analytical approach to enable unambiguous and sensitive evaluation of heterodimers directly from the mAb cocktail DP samples and, therefore, is of great utility to support mAb cocktail therapy development.

All references cited herein, including U.S. patent and applications are incorporated by reference in their entirety. It should be understood that the methods and systems of the present disclosure generally improve the sensitivity and specificity of detection, identification, and a quantitation of a molecule or molecules of interest, or fragments or multimers thereof, and are not limited to the mAb co-formulations of the working examples.

### EMBODIMENTS

The following list of embodiments is intended to complement, rather than displace or supersede, the previous descriptions.

### Embodiments Set I

Embodiment I-1. A method for identifying and/or quantifying at least one polypeptide or polypeptide multimer of interest, the method comprising:
subjecting a sample including at least one polypeptide or polypeptide multimer of interest to deglycosylation to form a deglycosylated sample;
subjecting said deglycosylated sample to size exclusion chromatography separation to form a separated sample; and
subjecting said separated sample to mass spectrometry analysis to identify and/or quantify said at least one polypeptide or polypeptide multimer of interest, wherein the method is performed under native conditions.

Embodiment I-2. The method of embodiment 1, wherein said size exclusion chromatography system and said mass spectrometer are connected by a splitter.

Embodiment I-3. The method of any of the preceding embodiments, wherein said splitter splits an eluate from said size exclusion chromatography system between said mass spectrometer and an ultraviolet detection system.

Embodiment I-4. The method of any of the preceding embodiments, wherein said at least one polypeptide or polypeptide multimer of interest comprises a homodimer and/or a heterodimer.

Embodiment I-5. The method of any of the preceding embodiments, wherein said at least one polypeptide or polypeptide multimer of interest is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof.

Embodiment I-6. The method of any of the preceding embodiments, wherein said sample is a co-formulation sample.

Embodiment I-7. The method of any of the preceding embodiments, wherein said sample includes two, three, or more antibodies.

Embodiment I-8. The method of any of the preceding embodiments, wherein said mass spectrometry analysis comprises nano-electrospray ionization mass spectrometry.

Embodiment I-9. The method of any of the preceding embodiments, wherein at least two polypeptides or polypeptide multimers of interest are identified or quantified, and said identifying and/or quantifying includes determining a relative abundance of each of said at least two polypeptides or polypeptide multimers of interest.

Embodiment I-10. The method of any of the preceding embodiments, further comprising quantifying an absolute percent of said at least two polypeptides or polypeptide multimers of interest in said sample.

Embodiment I-11. The method of any of the preceding embodiments, wherein said quantifying comprises multiplying said relative abundance determined in claim 9 by a total percent of said at least two polypeptides or polypeptide multimers of interest in said sample as measured using ultraviolet detection.

Embodiment I-12. The method of any of the preceding embodiments, wherein said size exclusion chromatography is high-throughput size exclusion chromatography.

Embodiment I-13. The method of any of the preceding embodiments, wherein said mass spectrometry is low-resolution mass spectrometry.

Embodiment I-14. The method of any of the preceding embodiments, wherein said sample is an immuno-depleted sample.

Embodiment I-15. The method of any of the preceding embodiments, wherein said immuno-depleted sample is formed by subjecting said sample to at least one immunoprecipitation step comprising at least one immunoprecipitation antibody that binds to at least one molecule in said sample that is not said at least one polypeptide or polypeptide multimer of interest.

Embodiment I-16. The method of any of the preceding embodiments, wherein said sample is a stressed sample.

Embodiment I-17. The method of any of the preceding embodiments, wherein said stress comprises storing said sample at a temperature from −80° C. to 40° C., about 5° C., about 25° C., or about 40° C.

Embodiment I-18. The method of any of the preceding embodiments, wherein said sample is stored at said temperature from 0 to 6 months, about 1 month, about 3 months, or about 6 months.

Embodiment I-19. A method for identifying and/or quantitating at least one antibody dimer in an antibody co-formulation, the method comprising:
subjecting an antibody co-formulation sample to deglycosylation to form a deglycosylated sample;
subjecting said deglycosylated sample to high-throughput size exclusion chromatography separation to form a separated sample; and
subjecting said separated sample to low-resolution mass spectrometry analysis to identify and/or quantify said at least one antibody dimer,
wherein the method is performed under native conditions and at least one splitter with at least three paths is used to couple said size exclusion chromatography system with said mass spectrometer and with an ultraviolet detector.

Embodiment I-20. The method of any of the preceding embodiments, wherein said antibody co-formulation sample comprises at least two antibodies or at least three antibodies.

Embodiment I-21. The method of any of the preceding embodiments, wherein said antibody co-formulation sample comprises at least one homodimer and at least one heterodimer.

Embodiment I-22. The method of any of the preceding embodiments, wherein said antibody co-formulation sample comprises at least two dimers, at least three dimers, at least four dimers, at least five dimers, or at least six dimers.

Embodiment I-23. The method of any of the preceding embodiments, wherein said antibody co-formulation sample is an immuno-depleted sample.

Embodiment I-24. The method of any of the preceding embodiments, wherein said immuno-depleted sample is formed by subjecting said sample to at least one immunoprecipitation step comprising at least one immunoprecipitation antibody that binds to at least one antibody in said sample.

### Embodiments Set II

Embodiment II-1. A method for identifying and/or quantifying at least one polypeptide or polypeptide multimer of interest, the method comprising:
subjecting a sample including at least one polypeptide or polypeptide multimer of interest to deglycosylation to form a deglycosylated sample;
subjecting said deglycosylated sample to size exclusion chromatography separation to form a separated sample; and
subjecting said separated sample to mass spectrometry analysis to identify and/or quantify said at least one polypeptide or polypeptide multimer of interest,
wherein the method is performed under native conditions.

Embodiment II-2. The method of embodiment 1, wherein said size exclusion chromatography system and said mass spectrometer are connected by a three-way splitter.

Embodiment II-3. The method of any of the preceding embodiments, wherein said three-way splitter splits an eluate from said size exclusion chromatography system between said mass spectrometer and an ultraviolet detection system.

Embodiment II-4. The method of any of the preceding embodiments, wherein said at least one polypeptide or polypeptide multimer of interest comprises a homodimer and/or a heterodimer.

Embodiment II-5. The method of any of the preceding embodiments, wherein said at least one polypeptide or polypeptide multimer of interest is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and a multimer thereof.

Embodiment II-6. The method of any of the preceding embodiments, wherein said sample is a co-formulation sample.

Embodiment II-7. The method of any of the preceding embodiments, wherein said sample includes two, three, or more antibodies.

Embodiment II-8. The method of any of the preceding embodiments, wherein the concentration of said antibody is about 0.5 mg/mL to about 100 mg/mL.

Embodiment II-9. The method of any of the preceding embodiments, wherein the concentration of said antibody is about 0.5 mg/mL to about 5 mg/mL.

Embodiment II-10. The method of any of the preceding embodiments, wherein said mass spectrometry analysis comprises nano-electrospray ionization mass spectrometry.

Embodiment II-11. The method of any of the preceding embodiments, wherein at least two polypeptides or polypeptide multimers of interest are identified or quantified, and said identifying and/or quantifying includes determining a relative abundance of each of said at least two polypeptides or polypeptide multimers of interest.

Embodiment II-12. The method of any of the preceding embodiments, further comprising quantifying an absolute percent of said at least two polypeptides or polypeptide multimers of interest in said sample.

Embodiment II-13. The method of any of the preceding embodiments, wherein said quantifying comprises multiplying said relative abundance determined in claim 9 by a total percent of said at least two polypeptides or polypeptide multimers of interest in said sample as measured using ultraviolet detection.

Embodiment II-14. The method of any of the preceding embodiments, wherein said size exclusion chromatography separation is performed with a flow rate of 0.2 mL/min.

Embodiment II-15. The method of any of the preceding embodiments, wherein a mobile phase for said size exclusion chromatography separation comprises ammonium acetate, ammonium bicarbonate, or ammonium formate, or combinations thereof.

Embodiment II-16. The method of any of the preceding embodiments, wherein a mobile phase for said size exclusion chromatography separation comprises ammonium acetate.

Embodiment II-17. The method of any of the preceding embodiments, wherein the concentration of ammonium acetate is 150 mM.

Embodiment II-18. The method of any of the preceding embodiments, wherein said size exclusion chromatography is high-throughput size exclusion chromatography.

Embodiment II-19. The method of any of the preceding embodiments, wherein said high-throughput size exclusion chromatography separation is performed with a 150 mm size exclusion chromatography column.

Embodiment II-20. The method of any of the preceding embodiments, wherein said mass spectrometry is low-resolution mass spectrometry.

Embodiment II-21. The method of any of the preceding embodiments, wherein the resolution setting for said low-resolution mass spectrometry is 6,250 (at m/z=200).

Embodiment II-22. The method of any of the preceding embodiments, wherein said sample is an immuno-depleted sample.

Embodiment II-23. The method of any of the preceding embodiments, wherein said immuno-depleted sample is formed by subjecting said sample to at least one immunoprecipitation step comprising at least one immunoprecipitation antibody that binds to at least one molecule in said sample that is not said at least one polypeptide or polypeptide multimer of interest.

Embodiment II-24. The method of any of the preceding embodiments, wherein said sample is a stressed sample.

Embodiment II-25. The method of any of the preceding embodiments, wherein said stress comprises storing said sample at a temperature from −80° C. to 40° C., about 5° C., about 25° C., or about 40° C.

Embodiment II-26. The method of any of the preceding embodiments, wherein said sample is stored at said temperature from 0 to 6 months, about 1 month, about 3 months, or about 6 months.

Embodiment II-27. The method of any of the preceding embodiments, wherein subjecting a sample including at least one polypeptide or polypeptide multimer of interest to deglycosylation comprises contacting said sample to PNGase F.

Embodiment II-28. A method for identifying and/or quantitating at least one antibody dimer in an antibody co-formulation, the method comprising:
subjecting an antibody co-formulation sample to deglycosylation to form a deglycosylated sample;
subjecting said deglycosylated sample to high-throughput size exclusion chromatography separation to form a separated sample; and
subjecting said separated sample to low-resolution mass spectrometry analysis to identify and/or quantify said at least one antibody dimer,
wherein the method is performed under native conditions and at least one splitter with at least three paths is used to couple said size exclusion chromatography system with said mass spectrometer and with an ultraviolet detector.

Embodiment II-29. The method of any of the preceding embodiments, wherein said antibody co-formulation sample comprises at least two antibodies or at least three antibodies.

Embodiment II-30. The method of any of the preceding embodiments, wherein the concentration of said antibody is about 0.5 mg/mL to about 100 mg/mL.

Embodiment II-31. The method of any of the preceding embodiments, wherein the concentration of said antibody is about 0.5 mg/mL to about 5 mg/mL.

Embodiment II-32. The method of any of the preceding embodiments, wherein said antibody co-formulation sample comprises at least one homodimer and at least one heterodimer.

Embodiment II-33. The method of any of the preceding embodiments, wherein said antibody co-formulation sample comprises at least two dimers, at least three dimers, at least four dimers, at least five dimers, or at least six dimers.

Embodiment II-34. The method of any of the preceding embodiments, wherein subjecting a sample including at least one polypeptide or polypeptide multimer of interest to deglycosylation comprises contacting the sample to PNGase F.

Embodiment II-35. The method of any of the preceding embodiments, wherein said antibody co-formulation sample is an immuno-depleted sample.

Embodiment II-36. The method of any of the preceding embodiments, wherein said immuno-depleted sample is formed by subjecting said sample to at least one immunoprecipitation step comprising at least one immunoprecipitation antibody that binds to at least one antibody in said sample.

Embodiment II-37. The method of any of the preceding embodiments, wherein said high-throughput size exclusion chromatography separation is performed with a flow rate of 0.6 mL/min.

Embodiment II-38. The method of any of the preceding embodiments, wherein said high-throughput size exclusion chromatography separation is performed with a 150 mm size exclusion chromatography column.

Embodiment II-39. The method of any of the preceding embodiments, wherein a mobile phase for said high-throughput size exclusion chromatography separation comprises ammonium acetate, ammonium bicarbonate, or ammonium formate, or combinations thereof.

Embodiment II-40. The method of any of the preceding embodiments, wherein a mobile phase for said high-throughput size exclusion chromatography separation comprises ammonium acetate.

Embodiment II-41. The method of any of the preceding embodiments, wherein the concentration of ammonium acetate is 150 mM.

Embodiment II-42. The method of any of the preceding embodiments, wherein said low-resolution mass spectrometry analysis comprises nano-electrospray ionization mass spectrometry.

Embodiment II-43. The method of any of the preceding embodiments, wherein the resolution setting for said low-resolution mass spectrometry analysis is 6,250 (at m/z=200).

### Embodiment Set III

Embodiment III-1. A method for identifying and/or quantifying a multimer of interest from an antibody preparation, the method comprising:
deglycosylating the antibody preparation to form a deglycosylated sample;
separating the deglycosylated sample by size exclusion chromatography separation to form a separated sample; and
analyzing the separated sample by nano-electrospray ionization (NSI) mass spectrometry analysis to identify and/or quantify the multimer of interest.

Embodiment III-2. The method of embodiment 1, wherein the multimer of interest comprises a homodimer and/or a heterodimer.

Embodiment III-3. The method of any of the preceding embodiments, wherein a monomer of the multimer of interest comprises an antibody, an antibody fragment, a fusion protein, a receptor fusion protein, an antibody-derived protein, an antigen-binding protein, or a variant or fragment thereof.

Embodiment III-4. The method of any of the preceding embodiments, wherein the antibody preparation comprises two or more antibodies.

Embodiment III-5. The method of any of the preceding embodiments, wherein the concentration of the two or more antibodies is about 0.5 mg/mL to about 100 mg/mL.

Embodiment III-6. The method of any of the preceding embodiments, further comprising quantifying a total percent abundance of the multimer of interest in the sample.

Embodiment III-7. The method of any of the preceding embodiments, wherein the size exclusion chromatography is a high-throughput size exclusion chromatography and the separating performed with a 100 mm-200 mm size exclusion chromatography column.

Embodiment III-8. The method of any of the preceding embodiments, wherein the mass spectrometry is low-resolution mass spectrometry comprising a resolution setting of 6,250 (at m/z=200).

Embodiment III-9. The method of any of the preceding embodiments, wherein the antibody preparation is immuno-depleted prior to deglycosylation by incubating the antibody preparation with at least one immunoprecipitation antibody that binds to at least one molecule in the antibody preparation that is not the multimer of interest.

Embodiment III-10. The method of any of the preceding embodiments, wherein the antibody preparation is stressed prior to deglycosylaton by storing the antibody preparation at a temperature from −80° C. to 40° C.

Embodiment III-11. The method of any of the preceding embodiments, wherein the antibody preparation is stored at the temperature from 0 to about 6 months.

Embodiment III-12. The method of any of the preceding embodiments, wherein the deglycosylation comprises incubating the antibody preparation with PNGase F.

Embodiment III-13. The method of any of the preceding embodiments, wherein the high-throughput size exclusion chromatography separation is performed at a flow rate of about 0.3 to about 0.8 mL/min.

Embodiment III-14. The method of any of the preceding embodiments, wherein a mobile phase for the size exclusion chromatography separation comprises about 50 mM to about 300 mM ammonium acetate.

What is claimed is:

1. A method for identifying and/or quantifying a multimer of interest from an antibody preparation, the method comprising:
deglycosylating the antibody preparation to form a deglycosylated sample;
separating the deglycosylated sample by size exclusion chromatography separation to form a separated sample; and
analyzing the separated sample by nano-electrospray ionization (NSI) mass spectrometry analysis to identify and/or quantify the multimer of interest.

2. The method of claim 1, wherein the multimer of interest comprises a homodimer and/or a heterodimer.

3. The method of claim 1, wherein a monomer of the multimer of interest comprises an antibody, an antibody fragment, a fusion protein, a receptor fusion protein, an antibody-derived protein, an antigen-binding protein, or a variant or fragment thereof.

4. The method of claim 1, wherein the antibody preparation comprises two or more antibodies.

5. The method of claim 4, wherein the concentration of the two or more antibodies is about 0.5 mg/mL to about 100 mg/mL.

6. The method of claim 1, further comprising quantifying a total percent abundance of the multimer of interest in the sample.

7. The method of claim 1, wherein the size exclusion chromatography is a high-throughput size exclusion chromatography and the separating performed with a 100-200 mm size exclusion chromatography column.

8. The method of claim 1, wherein the mass spectrometry is low-resolution mass spectrometry comprising a resolution setting of 6,250 (at m/z=200).

9. The method of claim 1, wherein the antibody preparation is immuno-depleted prior to deglycosylation by incubating the antibody preparation with at least one immunoprecipitation antibody that binds to at least one molecule in the antibody preparation that is not the multimer of interest.

10. The method of claim 1, wherein the antibody preparation is stressed prior to deglycosylaton by storing the antibody preparation at a temperature from −80° C. to 40° C.

11. The method of claim 10, wherein the antibody preparation is stored at the temperature from 0 to about 6 months.

12. The method of claim 1, wherein the deglycosylation comprises incubating the antibody preparation with PNGase F.

13. The method of claim 7, wherein the high-throughput size exclusion chromatography separation is performed at a flow rate of about 0.3 mL/min to about 0.8 mL/min.

14. The method of claim 1, wherein a mobile phase for the size exclusion chromatography separation comprises about 50 mM to about 300 mM ammonium acetate.
